SEQUENCE LISTING (18 sequences)

SEQ ID NO: 1 - protein, 121 aa, Artificial Sequence [sequence omitted]

SEQ ID NO: 2 - protein, 108 aa, Artificial Sequence
  Light Chain Variable Region
DIQMTQSPSSLSASVGDRVTITCRASQNIATDVAWYQQKPGKAPKLLIYSASFLYSGVPSRFSGSGSGTDFTLTISSLQPEDFATYYCQQSEPEPYTFGQGTKVEIKR

SEQ ID NO: 3 - protein, 7 aa, Artificial Sequence
  HVR-H1
LSGDWIH

SEQ ID NO: 4 - protein, 12 aa, Artificial Sequence
  HVR-H2
VGEISAAGGYTD

SEQ ID NO: 5 - protein, 14 aa, Artificial Sequence
  HVR-H3
ARESRVSFEAAMDY

SEQ ID NO: 6 - protein, 7 aa, Artificial Sequence
  HVR-L1
NIATDVA

SEQ ID NO: 7 - protein, 4 aa, Artificial Sequence
  HVR-L2
SASF

SEQ ID NO: 8 - protein, 7 aa, Artificial Sequence
  HVR-L3
SEPEPYT

SEQ ID NO: 9 - protein, 640 aa, Homo sapiens [sequence omitted]

SEQ ID NO: 10 - DNA, 1709 bp, Homo sapiens
cactggttgcggagtgagctgggcgcgcggcgcgcaggcgccctggggacccggtagcggcggtggcggtggcggcggtggcggtggctgcggcgacggcagaggcgaagggagccggatcgccgacctgagcgggaggcggcggtggcggccatggcggcagatggagagcgttccccgctgctgtctgagcccatcgacggtggcgcgggcggcaacggtttagtggggcccggcgggagtggggctgggcccgggggaggcctgaccccctccgcaccaccgtacggagccgcatttcccccgtttcccgaggggcatccagccgtgttgcctggggaggacccacccccctattcacccttaactagcccggacagtgggagtgcccctatgatcacctgccgagtctgccaatctctcatcaacgtggaaggcaagatgcatcagcatgtagtcaaatgtggtgtctgcaatgaagccaccccaatcaagaatgcacccccagggaaaaaatatgttcgatgcccctgtaactgtctccttatctgcaaagtgacatcccaacggattgcatgccctcggccctactgcaaaagaatcatcaacctggggcctgtgcatcccggacctctgagtccagaaccccaacccatgggtgtcagggttatctgtggacattgcaagaatacttttctgtggacagagttcacagaccgcactttggcacgttgtcctcactgcaggaaagtgtcatctattgggcgcagatacccacgtaagagatgtatctgctgcttcttgcttggcttgcttttggcagtcactgccactggccttgcctttggcacatggaagcatgcacggcgatatggaggcatctatgcagcctgggcatttgtcatcctgttggctgtgctgtgtttgggccgggctctttattgggcctgtatgaaggtcagccaccctgtccagaacttctcctgagcctgatgacccacagactgtgcctggcccctccctggtggggacagtgacactacgaagggagctggggtagttaaaggctcccggggcttctagaaggaagccaagcagctgccttccttttccctggggagaggtaggaaggaaccaggccctcacttaggtttggaggggcagataagagcactgctgaccatctgctttcctccaagggttgctgtgtctagggtgaagtaggcaaaacgttgcccttaaaactgggccctgaagacggttccagccttgtccttcctgtgtgctccctgagagccattcctgtcccttacacattccagggcagggtgggggtgggtagccctgggggttcccctccctcttgtgcaccattaggactttgctgctgctattgcacttcaccagaggttggctctggcctcagtaccctcagtctcctctccccacattgtgtcctgtgggggtggggtcagccgctgctctgtacagaaccacaggaactgatgtgtatataactatttaatgtgggatatgttcccctattcctgtatttcccttaattcctcctcccgaccttttttacccccccagttgcagtatttaactgggctgggtagggttgctcagtctttgggggaggttagggacttatcctgtgcttgtaaataaataaggtcatgactctacgcttaaaaaaaaaaaaaaaa

SEQ ID NO: 11 - DNA, 1730 bp, Homo sapiens
cactggttgcggagtgagctgggcgcgcggcgcgcaggcgccctggggacccggtagcggcggtggcggtggcggcggtggcggtggctgcggcgacggcagaggcgaagggagccggatcgccgacctgagcgggaggcggcggtggcggccatggcggcagatggagagcgttccccgctgctgtctgagcccatcgacggtggcgcgggcggcaacggtttagtggggcccggcgggagtggggctgggcccgggggaggcctgaccccctccgcaccaccgtacggagccggtaaacatgccccgccccaggcatttcccccgtttcccgaggggcatccagccgtgttgcctggggaggacccacccccctattcacccttaactagcccggacagtgggagtgcccctatgatcacctgccgagtctgccaatctctcatcaacgtggaaggcaagatgcatcagcatgtagtcaaatgtggtgtctgcaatgaagccaccccaatcaagaatgcacccccagggaaaaaatatgttcgatgcccctgtaactgtctccttatctgcaaagtgacatcccaacggattgcatgccctcggccctactgcaaaagaatcatcaacctggggcctgtgcatcccggacctctgagtccagaaccccaacccatgggtgtcagggttatctgtggacattgcaagaatacttttctgtggacagagttcacagaccgcactttggcacgttgtcctcactgcaggaaagtgtcatctattgggcgcagatacccacgtaagagatgtatctgctgcttcttgcttggcttgcttttggcagtcactgccactggccttgcctttggcacatggaagcatgcacggcgatatggaggcatctatgcagcctgggcatttgtcatcctgttggctgtgctgtgtttgggccgggctctttattgggcctgtatgaaggtcagccaccctgtccagaacttctcctgagcctgatgacccacagactgtgcctggcccctccctggtggggacagtgacactacgaagggagctggggtagttaaaggctcccggggcttctagaaggaagccaagcagctgccttccttttccctggggagaggtaggaaggaaccaggccctcacttaggtttggaggggcagataagagcactgctgaccatctgctttcctccaagggttgctgtgtctagggtgaagtaggcaaaacgttgcccttaaaactgggccctgaagacggttccagccttgtccttcctgtgtgctccctgagagccattcctgtcccttacacattccagggcagggtgggggtgggtagccctgggggttcccctccctcttgtgcaccattaggactttgctgctgctattgcacttcaccagaggttggctctggcctcagtaccctcagtctcctctccccacattgtgtcctgtgggggtggggtcagccgctgctctgtacagaaccacaggaactgatgtgtatataactatttaatgtgggatatgttcccctattcctgtatttcccttaattcctcctcccgaccttttttacccccccagttgcagtatttaactgggctgggtagggttgctcagtctttgggggaggttagggacttatcctgtgcttgtaaataaataaggtcatgactctacgcttaaaaaaaaaaaaaaaa

SEQ ID NO: 12 - DNA, 2639 bp, Homo sapiens
gagatctaggaagtcgcttctttttctggtagaaggcggggttctcctcgtacgctgcggagtctctgcggggtgtagaccggaatcctgctgacgggcagagtggatcagggagggagggtcgagacacggtggctgcaggtctgagacaaggctgctccgaggtagtagctctcttgcctggaggtggccattcattcctggagtgctgctgaggagcgagggcccatctggggtctctggaagtcggtgcccaggcctgaaggatagccccccttgcgcttccctgggctgcggccggccttctcagaacgaagggcgtccttccaccccgcggcgcaggtgaccgctgccatggcttttccccatcggccggacgcccctgagctgcctgacttctccatgctgaagaggctggctcgagaccagctcatctatctgctggagcagcttcctggaaaaaaggatttattcattgaggcagatctcatgagccctttggatcgaattgccaatgtctccatcctgaagcaacacgaagtagacaagctatacaaggtggagaacaagccagccctcagctccaatgaacaattgtgcttcttggtcagaccccgcatcaagaatatgcgatacattgccagtcttgtcaatgctgacaaattggctggccgaactcgcaaatacaaagtgatcttcagccctcaaaagttctatgcgtgtgagatggtgcttgaggaagagggaatctatggagatgtgagctgtgatgaatgggccttctctttgctgcctcttgatgtggatctgctgagcatggaactaccagaatttttcagggattactttctggaaggagatcagcgttggatcaacactgtagctcaggccttacaccttctcagcactctctatggaccctttccaaactgctatggaattggcaggtgcgccaagatggcatatgaattgtggaggaacctggaggaggaggaggatggcgaaaccaagggccgaaggccagagattggacatatctttctcttggacagagatgtggactttgtgacagcactttgctcccaagtggtttatgagggcctagtagatgacaccttccgcatcaagtgtgggagtgtcgactttggcccagaagtcacatcctctgacaagagcctgaaggtgctactcaatgccgaggacaaggtgtttaatgagattcggaacgagcacttctccaatgtctttggcttcttgagccagaaggcccggaacttgcaggcccagtatgatcgccggagaggcatggacattaagcagatgaagaatttcgtgtcccaggagctcaagggcctgaaacaggagcaccgcctgctgagtctccatattggggcctgtgaatccatcatgaagaagaaaaccaagcaggatttccaggagctaatcaagactgagcatgcactgctagaggggttcaacatccgggagagcaccagctacattgaggaacacatagaccggcaggtgtcgcctatagaaagcctgcgcctcatgtgccttttgtccatcactgagaatggtttgatccccaaggattaccgatctctgaaaacacagtatctgcagagctatggccctgagcacctgctaaccttctccaatctgcgaagagctgggctcctaacggagcaggcccccggggacaccctcacagccgtggagagtaaagtgagcaagctggtgaccgacaaggctgcaggaaagattactgatgccttcagttctctggccaagaggagcaattttcgtgccatcagcaaaaagctgaatttgatcccacgtgtggacggcgagtatgatctgaaagtgccccgagacatggcttacgtcttcggtggtgcttatgtgcccctgagctgccgaatcattgagcaggtgctagagcggcgaagctggcagggccttgatgaggtggtacggctgctcaactgcagtgactttgcattcacagatatgactaaggaagacaaggcttccagtgagtccctgcgcctcatcttggtggtgttcttgggtggttgtacattctctgagatctcagccctccggttcctgggcagagagaaaggctacaggttcattttcctgacgacagcagtcacaaacagcgctcgccttatggaggccatgagtgaggtgaaagcctgatgtttttcccggccagtgttgacatcttccctgaacacattcctcagtgagatgcaggcatctggcacccagctgctataaccaagtgtccaccaactacctgctaagagccgggagcatggaacgtgttgggatttagagaacattatctgagaaaagagttcacttcctgctcccaggatatttctcttttctgtttatgaagtacaacccatgctgctaagatgcgagcaggaagaggcatcctttgctaaatcctgtttgaatgtcattgtaaataaagcctctgctctcagatgtaatacgttggtcgttctggacacgcacttccatctttattagtgaaagcccattggagcttgctacaggggcttcctgtgcttcttggcctggactttgtagtggctccaggcctggtctggttcttccct

SEQ ID NO: 13 - DNA, 20 bp, Artificial Sequence
  PCR Primer
ggcctcagtaccctcagtct

SEQ ID NO: 14 - DNA, 16 bp, Artificial Sequence
  PCR primer
agagcagcggctgacc

SEQ ID NO: 15 - DNA, 16 bp, Artificial Sequence
  Probe
ccccacaggacacaat

SEQ ID NO: 16 - DNA, 21 bp, Artificial Sequence
  PCR Primer
ggctcgagaccagctcatcta

SEQ ID NO: 17 - DNA, 25 bp, Artificial Sequence
  PCR Primer
gagatctgcctcaatgaataaatcc

SEQ ID NO: 18 - DNA, 14 bp, Artificial Sequence
  Probe
tggagcagcttcct

(19) United States
(12) Patent Application Publication
(10) Pub. No.: US 2013/0259867 A1
(43) Pub. Date: Oct. 3, 2013

(54) DIAGNOSIS AND TREATMENTS RELATING TO HER3 INHIBITORS

(71) Applicant: GENENTECH, INC., South San Francisco, CA (US)

(72) Inventors: Lukas Amler, Foster City, CA (US); David Shames, San Francisco, CA (US)

(73) Assignee: GENENTECH, INC., South San Francisco, CA (US)

(21) Appl. No.: 13/835,532

(22) Filed: Mar. 15, 2013

Related U.S. Application Data
(60) Provisional application No. 61/616,241, filed on Mar. 27, 2012.

(51) Int. Cl. G01N 33/68

(52) U.S. Cl. CPC G01N 33/6893
USPC 424/136.1; 424/172.1; 435/7.1; 435/6.12

(57) ABSTRACT

The present application describes the use of NRG1 overexpression as a selection criterion for treating cancer patients with a HER3 inhibitor, such as a bispecific HER3/EGFR inhibitor, and methods of treating those patients.

### CROSS REFERENCE TO RELATED APPLICATIONS

This application claims the benefit of priority to U.S. Patent Application 61/616,241, filed Mar. 27, 2012, the entire contents of which is incorporated herein by reference in its entirety.

### FIELD OF THE INVENTION

The present application relates to the field of cancer therapy and methods of selecting cancer patients for treatment with a HER3 inhibitor.

### SEQUENCE LISTING

The present application contains a Sequence Listing which has been submitted in ASCII format via EFS-Web and is hereby incorporated by reference in its entirety. Said ASCII copy, created on Mar. 11, 2013, is named P4884R1US_ST25.txt and is 3,192,990 bytes in size.

### BACKGROUND OF THE INVENTION

The HER family of receptor tyrosine kinases are important mediators of cell growth, differentiation and survival. The receptor family includes four distinct members including epidermal growth factor receptor (EGFR, ErbB1, or HER1), HER2 (ErbB2 or p185^{neu}), HER3 (ErbB3) and HER4 (ErbB4 or tyro2).

Therapeutics that target the HER pathway are presently in use in treating diseases such as breast cancer, non-small cell lung cancer, colorectal cancer, head and neck cancer and pancreatic cancer.

EGFR is bound by six different ligands; epidermal growth factor (EGF), transforming growth factor alpha (TGF-α), amphiregulin, heparin binding epidermal growth factor (HB-EGF), betacellulin and epiregulin (Groenen et al. Growth Factors, 11:235-257 (1994)).

Neuregulin is a ligand for the Her3 and Her4 receptor tyrosine kinases. There are four known members of the neuregulin family, NRG1, NRG2, NRG3, and NRG4 (Falls, D. L., Ex Cell Res, 284:14-30 (2003); Hirsch and Wu (2007). Expert Reviews, Vol. 7, 147-157). The NRG1 transcript undergoes extensive alternative splicing resulting in at least 15 different isoforms. All active isoforms share an EGF-like domain that is necessary and sufficient for activity (Holmes, W. E., et al., Science, 256:1205-1210 (1992); Yarden, Y., and Peles, E., Biochemistry 30: 3543-3550 (1991)).

Approximately 52,140 new cases of squamous cell carcinoma of the head and neck (HNSCC) were diagnosed and 11,460 people are estimated to have died from this disease in the United States last year (1). Curative interventions for HNSCC include surgery, radiation, and combined radio-chemotherapy. The overall 5-year relative survival rate for primary HNSCC is approximately 60%. However, the 5-year relative survival rate is only 35% for patients diagnosed with metastatic disease (2). The poor outcomes in patients with advanced HNSCC clearly indicate the need for more effective therapies in this population (3).

Signaling through the epidermal growth factor receptor (EGFR) pathway is a major driver of HNSCC (4). EGFR is overexpressed in up to 90% of all HNSCC (5, 6). EGFR inhibition with cetuximab has proven to be a successful therapeutic strategy, albeit with somewhat limited long-term clinical benefits due to intrinsic or acquired resistance (7). Tyrosine kinase inhibitors (TKIs), such as erlotinib, gefitinib, and lapatinib, that target EGFR and or HER2 have been investigated in clinical studies of HNSCC but have not demonstrated a survival advantage in randomized trials (8-10).

NRG1 autocrine signaling has been shown to regulate lung epithelial cell proliferation (Jinbo, et al., Am. J. Respir. Cell Mol. Biol. 27:306-313 (2002) and to play a role in human lung development (Patel, et al, Am. J. Resp Cell Mol Bio, 22: 432-440 (2000)) and has been implicated in insensitivity of NSCLC to EGFR inhibitors (Zhou, et al., Cancer cell 10:39-50 (2006)). Preclinical studies suggested that certain cancer cell lines are driven by an autocrine-signaling loop in which NRG1 promotes malignancy by engaging the HER2 kinase. (Wilson, et al., Cancer Cell, 20:158-173 (2011)).

### SUMMARY OF THE INVENTION

One aspect of the invention provides a method of treating a type of cancer in a patient comprising administering a therapeutically effective amount of a HER3 inhibitor to the patient, where the patient, prior to administration of the HER3 inhibitor, was diagnosed with a cancer which overexpresses NRG1. NRG1 overexpression is indicative of therapeutic responsiveness by the subject to the HER3 inhibitor. In one embodiment, the patient was diagnosed with a cancer expressing NRG1 at a level higher than the median level for NRG1 expression in the cancer type. In certain embodiments, the patient was diagnosed with a cancer expressing NRG1 at a level which is at the 60th percentile or higher, 75th percentile or higher, or 80th percentile or higher for NRG1 expression in the cancer type. In one embodiment, the type of cancer is one which exhibits a bimodal expression profile consisting of an overexpression mode and a lack of overexpression mode. In one embodiment, the inflection point of the bimodal expression profile is 1.5 as measured on a linear scale. In one embodiment, the type of cancer is one which exhibits autocrine neuregulin-induced signaling, such HNSCC.

In one embodiment, the HER3 inhibitor inhibits NRG1 binding to HER3. In one embodiment, the HER3 inhibitor is an antibody. In one embodiment, the HER3 inhibitor is a bispecific HER3/EGFR inhibitor. In one embodiment, the bispecific HER3/EGFR inhibitor is a bispecific antibody which comprises an antigen binding domain that specifically binds to HER3 and EGFR. In one embodiment, the bispecific antibody comprises the HVR sequences of the heavy chain variable domain of SEQ ID NO: 1 and the HVR sequences of the light chain variable domain sequence of SEQ ID NO: 2. In one embodiment, the bispecific antibody comprises the heavy chain variable domain of SEQ ID NO: 1 and the light chain variable domain sequence of SEQ ID NO: 2.

In one embodiment, the diagnosis comprised determining the expression level of NRG1 in a sample from the patient's cancer and quantifying the expression level of NRG1 in the sample relative to the expression level of one or both of AL-137727 and VPS33B in the sample.

Another aspect of the invention provides a method of treating head and neck squamous cell carcinoma (HNSCC) in a patient comprising administering a therapeutically effective amount of a bispecific HER3/EGFR inhibitor to the patient, wherein the patient, prior to administration of the bispecific HER3/EGFR inhibitor, was diagnosed with a HNSCC which overexpresses NRG1. NRG1 overexpression is indicative of therapeutic responsiveness by the subject to the bispecific HER3/EGFR inhibitor. In one embodiment, the bispecific HER3/EGFR inhibitor is a bispecific antibody which comprises an antigen binding domain that specifically binds to HER3 and EGFR. In one embodiment, the bispecific antibody comprises the HVR sequences of the heavy chain variable domain of SEQ ID NO: 1 and the HVR sequences of the light chain variable domain sequence of SEQ ID NO: 2. In one embodiment, the bispecific antibody comprises the heavy chain variable domain of SEQ ID NO: 1 and the light chain variable domain sequence of SEQ ID NO: 2.

Another aspect of the invention provides a method for selecting a therapy for a patient with a type of cancer which exhibits autocrine neuregulin-induced signaling comprising determining neuregulin 1 (NRG1) expression in a cancer sample from the patient and selecting a HER3 inhibitor for therapy if the cancer sample overexpresses NRG1. In one embodiment, the cancer sample expresses NRG1 at a level higher than the median level for NRG1 expression in the cancer type. In certain embodiments, the cancer sample expresses NRG1 at a level which is at the 60th percentile or higher, 75th percentile or higher, or 80th percentile or higher for NRG1 expression in the cancer type. In one embodiment, the type of cancer is one which exhibits a bimodal expression profile consisting of an overexpression mode and a lack of overexpression mode. In one embodiment, the inflection point of the bimodal expression profile is 1.5 as measured on a linear scale. In one embodiment, the type of cancer is one which exhibits autocrine neuregulin-induced signaling, such HNSCC. In one embodiment, the method further comprises administering a therapeutically effective amount of the HER3 inhibitor to the patient. In one embodiment, the type of cancer is HNSCC. In one embodiment, the HER3 inhibitor inhibits NRG binding to HER3. In one embodiment, the HER3 inhibitor is an antibody. In one embodiment, the HER3 inhibitor is a bispecific HER3/EGFR inhibitor. In one embodiment, the bispecific HER3/EGFR inhibitor is a bispecific antibody which comprises an antigen binding domain that specifically binds to HER3 and EGFR. In one embodiment, the bispecific antibody comprises the HVR sequences of the heavy chain variable domain of SEQ ID NO: 1 and the HVR sequences of the light chain variable domain sequence of SEQ ID NO: 2. In one embodiment, the bispecific antibody comprises the heavy chain variable domain of SEQ ID NO: 1 and the light chain variable domain sequence of SEQ ID NO: 2.

In one embodiment, the determination of NRG1 expression comprises determining the expression level of NRG1 in a sample from the patient's cancer and quantifying the expression level of NRG1 in the sample relative to the expression level of one or both of AL-137727 and VPS33B in the sample.

Another aspect of the invention provides a method for selecting a therapy for a patient with a head and neck squamous cell carcinoma (HNSCC) comprising determining neuregulin 1 (NRG1) expression in a HNSCC sample from the patient and selecting a bispecific HER3/EGFR inhibitor as the therapy if the HNSCC sample overexpresses NRG1. In one embodiment, the method further comprises administering a therapeutically effective amount of the bispecific HER3/EGFR inhibitor inhibitor to the patient. In one embodiment, the bispecific HER3/EGFR inhibitor is a bispecific antibody which comprises an antigen binding domain that specifically binds to HER3 and EGFR. In one embodiment, the bispecific antibody comprises the HVR sequences of the heavy chain variable domain of SEQ ID NO: 1 and the HVR sequences of the light chain variable domain sequence of SEQ ID NO: 2. In one embodiment, the bispecific antibody comprises the heavy chain variable domain of SEQ ID NO: 1 and the light chain variable domain sequence of SEQ ID NO: 2. In one embodiment, the determination of NRG1 expression comprises determining the expression level of NRG1 in a sample from the patient's cancer and quantifying the expression level of NRG1 in the sample relative to the expression level of one or both of AL-137727 and VPS33B in the sample.

Another aspect of the invention provides a method for advertising a HER3 inhibitor or a pharmaceutically acceptable composition thereof comprising promoting, to a target audience, the use of the HER3 inhibitor or pharmaceutical composition thereof for treating a patient population with a type of cancer, where the patient's cancer overexpresses NRG1. In one embodiment, the HER3 inhibitor inhibits NRG binding to HER3. In one embodiment, the HER3 inhibitor is an antibody. In one embodiment, the HER3 inhibitor is a bispecific HER3/EGFR inhibitor. In one embodiment, the bispecific HER3/EGFR inhibitor is a bispecific antibody which comprises an antigen binding domain that specifically binds to HER3 and EGFR. In one embodiment, the bispecific antibody comprises the HVR sequences of the heavy chain variable domain of SEQ ID NO: 1 and the HVR sequences of the light chain variable domain sequence of SEQ ID NO: 2. In one embodiment, the bispecific antibody comprises the heavy chain variable domain of SEQ ID NO: 1 and the light chain variable domain sequence of SEQ ID NO: 2.

Another aspect of the invention provides a method for advertising a bispecific HER3/EGFR inhibitor or a pharmaceutically acceptable composition thereof comprising promoting, to a target audience, the use of the bispecific HER3/EGFR inhibitor or pharmaceutical composition thereof for treating a patient population with a HNSCC, where the patient's HNSCC overexpresses NRG1. In one embodiment, the bispecific HER3/EGFR inhibitor is a bispecific antibody which comprises an antigen binding domain that specifically binds to HER3 and EGFR. In one embodiment, the bispecific antibody comprises the HVR sequences of the heavy chain variable domain of SEQ ID NO: 1 and the HVR sequences of the light chain variable domain sequence of SEQ ID NO: 2. In one embodiment, the bispecific antibody comprises the heavy chain variable domain of SEQ ID NO: 1 and the light chain variable domain sequence of SEQ ID NO: 2.

Another aspect of the invention provides a method of quantifying NRG1 expression level in a cancer sample comprising determining the expression level of NRG1 in the sample, and quantifying the expression level of NRG1 in the sample relative to the expression level of an internal reference gene or genes in the sample. In one embodiment, the reference gene or genes is one or both of AL-137727 and VPS33B. In one embodiment, the sample is from a cancer that exhibits autocrine neuregulin-induced signaling, such as head and neck squamous cell carcinoma (HNSCC). In one specific embodiment, of this method, the expression level of NRG1 and the expression level of the one or both of AL-137727 and VPS33B is determined using polymerase chain reaction (PCR). In one embodiment, the PCR used in the method is quantitative real time polymerase chain reaction (qRT-PCR). In another embodiment, the expression level of NRG1 is determined using immunohistochemistry (IHC) or ELISA. In another embodiment, the expression level of NRG1 is determined using immunohistochemistry (IHC) or ELISA. In another embodiment, the expression level of NRG1 is determined by direct RNA sequencing. In another embodiment, the expression level of NRG1 is determined using RNA in situ hybridization.

### BRIEF DESCRIPTION OF THE DRAWINGS

FIG. 1 is a graph demonstrating that the MEHD7945A antibody binds to both HER3-ECD and EGFR-ECD.

FIGS. 2A and B are graphs demonstrating that MEHD7945A inhibits EGFR and HER2/HER3-dependent signaling.

FIG. 3 is a graph showing inhibition of tumor growth in FaDu cancer model by MEHD7945A.

FIG. 4 is a summary of the tumor growth inhibitory effect of MEHD7945A compared to cetuximab or anti-HER3 in numerous murine xenograft models.

FIG. 5 shows NRG1 (A) and HER3 (B) expression levels in cancer types as determined by qRT-PCR.

FIG. 6 shows the biomodal distribution of NRG1 expression in HNSCC as compared to other cancer types.

FIG. 7 shows the bimodal distribution of NRG1 expression in HNSCC when plotted on a log₁₀ scale. The dotted line indicates the inflection point between the overexpression and lack of overexpression modes of NRG1 expression in HNSCC, which is set at 0.3689 on the logarithm scale, corresponding to approximately 1.50 on the linear scale.

FIG. 8 shows the results of an IP-western blot analysis for pHER3 and pTyr in fresh frozen tumor specimens from patients with therapy naïve SCHNN. MCF7 is a negative control; MCF7+NRG and PCI6A are positive controls. The controls for the pTyr blot were run separately whereas the controls for the pHER3 were run concurrently.

FIG. 9 shows the results of a qRT-PCR assay indicating that high NRG1 expression is associated with pHER3 differential activation of HER3 signaling in 23 SCHNN tumors (18/19 overlap with the IP-western). Black lines below the x-axis indicate the tumors with detectable pHER3 by IP-western.

FIG. 10 is a table summarizing pathologic and demographic variables of patients and their tumors used in the analysis.

FIG. 11 shows qRT-PCR analysis comparing the levels of NRG1 expression in unmatched primary and recurrent HNSCC specimens.

FIG. 12 shows qRT-PCR analysis comparing the levels of NRG1 expression in matched primary and recurrent HNSCC.

FIG. 13 is a table showing comparisons between matched therapy-naïve and post therapy HNSCCs.

FIG. 14 is a table showing changes in NRG1 or HER3 expression and autocrine biology between matched therapy naïve and post-chemotherapy HNSCC samples. Definiens software was used to detect expression of NRG1 or HER3 or both transcripts within counterstained nuclei.

FIG. 15 shows pairwise analysis of RNA-ISH and qRT-PCR of NRG1 (A) and HER3 (B) in primary and recurrent SCHNNs. Spearman rank correlations and p-values are shown on the graphs.

FIG. 16 shows NRG1 expression levels of HNSCC and CRC patients.

FIG. 17 shows NRG1 expression levels in primary & recurrent HNSCC as compared to NRG1 expression levels of HNSCC patients in Phase 1 study.

FIG. 18 shows the amino acid sequence of antibody MEHD7945A (SEQ ID NOs: 1 and 2).

### DETAILED DESCRIPTION OF THE PREFERRED EMBODIMENTS

### I. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

Before the present methods, kits and uses therefore are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise.

Throughout this specification and claims, the word “comprise,” or variations such as “comprises” or “comprising,” will be understood to imply the inclusion of a stated integer or group of integers but not the exclusion of any other integer or group of integers.

The term “antibody” herein is used in the broadest sense and specifically covers monoclonal antibodies, polyclonal antibodies, multispecific antibodies, and antibody fragments so long as they exhibit the desired biological activity. The term “multispecific antibody” is used in the broadest sense and specifically covers an antibody comprising an antigen-binding domain that has polyepitopic specificity (i.e., is capable of specifically binding to two, or more, different epitopes on one biological molecule or is capable of specifically binding to epitopes on two, or more, different biological molecules). One specific example of an antigen-binding domain is a V_{H}V_{L} unit comprised of a heavy chain variable domain (V_{H}) and a light chain variable domain (V_{L}). Such multispecific antibodies include, but are not limited to, full length antibodies, antibodies having two or more V_{L} and V_{H} domains, antibody fragments such as Fab, Fv, dsFv, scFv, diabodies, bispecific diabodies and triabodies, antibody fragments that have been linked covalently or non-covalently. A “bispecific antibody” is a multispecific antibody comprising an antigen-binding domain that is capable of specifically binding to two different epitopes on one biological molecule or is capable of specifically binding to epitopes on two different biological molecules. The bispecific antibody is also referred to herein as having “dual specificity” or as being “dual specific”.

In certain embodiments, an antibody of the invention has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M) for its target HER or HERs.

The basic 4-chain antibody unit is a heterotetrameric glycoprotein composed of two identical light (L) chains and two identical heavy (H) chains (an IgM antibody consists of 5 of the basic heterotetramer units along with an additional polypeptide called J chain, and therefore contains 10 antigen-binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has, at the N-terminus, a variable domain (V_{H}) followed by three constant domains (C_{H}) for each of the α and γ chains and four C_{H} domains for μ and ε isotypes. Each L chain has, at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, γ, ε, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen-binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called hypervariable regions” or HVR. The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region,” “HVR,” or “HV,” when used herein refers to the regions of an antibody variable domain which are hypervariable in sequence and/or form structurally defined loops. Generally, antibodies comprise six HVRs; three in the VH(HVR-H1, HVR-H2, HVR-H3), and three in the VL (HVR-L1, HVR-L2, HVR-L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

HVRs generally comprise amino acid residues from the hypervariable loops and/or from the “complementarity determining regions” (CDRs), the latter being of highest sequence variability and/or involved in antigen recognition. A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 47-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see WO 2006/073941).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein.

An “affinity matured” antibody is one with one or more alterations in one or more HVRs or framework region thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies may be produced using certain procedures known in the art. For example, Marks et al. Bio/Technology 10:779-783 (1992) describes affinity maturation by VH and VL domain shuffling. Random mutagenesis of HVR and/or framework residues is described by, for example, Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “monoclonal antibody” as used herein refers to an antibody from a population of substantially homogeneous antibodies, i.e., the individual antibodies comprising the population are substantially similar and bind the same epitope(s), except for possible variants that may arise during production of the monoclonal antibody, such variants generally being present in minor amounts. Such monoclonal antibody typically includes an antibody comprising a variable region that binds a target, wherein the antibody was obtained by a process that includes the selection of the antibody from a plurality of antibodies. For example, the selection process can be the selection of a unique clone from a plurality of clones, such as a pool of hybridoma clones, phage clones or recombinant DNA clones. It should be understood that the selected antibody can be further altered, for example, to improve affinity for the target, to humanize the antibody, to improve its production in cell culture, to reduce its immunogenicity in vivo, to create a multispecific antibody, etc., and that an antibody comprising the altered variable region sequence is also a monoclonal antibody of this invention. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34):12467-12472 (2004); and Lee et al. J. Immunol. Methods 284(1-2):119-132 (2004) and technologies for producing human or human-like antibodies from animals that have parts or all of the human immunoglobulin loci or genes encoding human immunoglobulin sequences (see, e.g., WO98/24893, WO/9634096, WO/9633735, and WO/91 10741, Jakobovits et al., Proc. Natl. Acad. Sci. USA, 90:2551 (1993); Jakobovits et al., Nature, 362:255-258 (1993); Bruggemann et al., Year in Immuno, 7:33 (1993); U.S. Pat. Nos. 5,545,806, 5,569,825, 5,591,669 (all of GenPharm); 5,545,807; WO 97/17852, U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol, 13: 65-93 (1995).

An “intact” antibody is one which comprises an antigen-binding site as well as a C_{L} and at least heavy chain constant domains, C_{H}1, C_{H}2, and C_{H}3. The constant domains can be native sequence constant domains (e.g., human native sequence constant domains) or amino acid sequence variant thereof. Preferably, the intact antibody has one or more effector functions.

“Antibody fragments” comprise a portion of an intact antibody, preferably the antigen-binding or variable region of the intact antibody. Examples of antibody fragments include Fv, Fab, Fab′, F(ab′)₂, Fab′-SH; diabodies; linear antibodies (see U.S. Pat. No. 5,641,870, Example 2; Zapata et al., Protein Eng. 8(10): 1057-1062 (1995)); single-chain antibody molecules (e.g. scFv). While in the present description, and throughout the specification, reference is made to antibodies and various properties of antibodies, the same disclosure also applies to functional antibody fragments, e.g. dual action Fab fragments.

The expression “linear antibodies” generally refers to the antibodies described in Zapata et al., Protein Eng., 8(10):1057-1062 (1995). These antibodies comprise a pair of tandem Fd segments (V_{H}-C_{H}1-V_{H}-C_{H}1) which, together with complementary light chain polypeptides, form a pair of antigen-binding regions. In a preferred embodiment, the fragment is “functional,” i.e. qualitatively retains the ability of the corresponding intact antibody to bind to the target HER receptor and, if the intact antibody also inhibits HER activation or function, qualitatively retains such inhibitory property as well. Qualitative retention means that the activity in kind is retained, but the degree of binding affinity and/or activity might differ.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region; this region is also the part recognized by Fc receptors (FcR) found on certain types of cells.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen-binding and confer antigen-binding specificity to the antibody. However, even a single variable domain (or half of an Fv comprising only three HVRs specific for an antigen) has the ability to recognize and bind antigen, although often at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen-binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Diabodies are described more fully in, for example, EP 404,097; WO 93/11161; and Hollinger et al., Proc. Natl. Acad. Sci. USA, 90:6444-6448 (1993).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The term “chimeric” antibody refers to an antibody in which a portion of the heavy and/or light chain is derived from a particular source or species, while the remainder of the heavy and/or light chain is derived from a different source or species.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

An antibody of this invention “which binds” an antigen of interest is one that binds the antigen with sufficient affinity such that the antibody is useful as a diagnostic and/or therapeutic agent in targeting a protein or a cell or tissue expressing the antigen. With regard to the binding of a antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess non-labeled target. In one particular embodiment, “specifically binds” refers to binding of an antibody to its specified target HER receptors and not other specified non-target HER receptors. For example, the antibody specifically binds to EGFR and HER3 but does not specifically bind to HER2 or HER4, or the antibody specifically binds to EGFR and HER2 but does not specifically bind to HER3 or HER4, or the antibody specifically binds to EGFR and HER4 but does not specifically bind to HER2 or HER3.

A “HER receptor” is a receptor protein tyrosine kinase which belongs to the HER receptor family and includes EGFR (ErbB1, HER1), HER2 (ErbB2), HER3 (ErbB3) and HER4 (ErbB4) receptors. The HER receptor will generally comprise an extracellular domain, which may bind an HER ligand and/or dimerize with another HER receptor molecule; a lipophilic transmembrane domain; a conserved intracellular tyrosine kinase domain; and a carboxyl-terminal signaling domain harboring several tyrosine residues which can be phosphorylated. The HER receptor may be a “native sequence” HER receptor or an “amino acid sequence variant” thereof. Preferably the HER receptor is a native sequence human HER receptor.

The “HER pathway” refers to the signaling network mediated by the HER receptor family.

The terms “ErbB1”, “HER1”, “epidermal growth factor receptor” and “EGFR” are used interchangeably herein and refer to EGFR as disclosed, for example, in Carpenter et al. Ann. Rev. Biochem. 56:881-914 (1987), including naturally occurring mutant forms thereof (e.g. a deletion mutant EGFR as in Ullrich et al, Nature (1984) 309:418425 and Humphrey et al. PNAS (USA) 87:4207-4211 (1990)), as well we variants thereof, such as EGFRvIII. Variants of EGFR also include deletional, substitutional and insertional variants, for example those described in Lynch et al (New England Journal of Medicine 2004, 350:2129), Paez et al (Science 2004, 304:1497), and Pao et al (PNAS 2004, 101:13306).

Herein, “EGFR extracellular domain” or “EGFR ECD” refers to a domain of EGFR that is outside of a cell, either anchored to a cell membrane, or in circulation, including fragments thereof. In one embodiment, the extracellular domain of EGFR may comprise four domains: “Domain I” (amino acid residues from about 1-158, “Domain II” (amino acid residues 159-336), “Domain III” (amino acid residues 337-470), and “Domain IV” (amino acid residues 471-645), where the boundaries are approximate, and may vary by about 1-3 amino acids.

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (GenBank accession number X03363). The term “erbB2” refers to the gene encoding human HER2 and “neu” refers to the gene encoding rat p185^{neu}. Preferred HER2 is native sequence human HER2.

Herein, “HER2 extracellular domain” or “HER2ECD” refers to a domain of HER2 that is outside of a cell, either anchored to a cell membrane, or in circulation, including fragments thereof. In one embodiment, the extracellular domain of HER2 may comprise four domains: “Domain I” (amino acid residues from about 1-195, “Domain II” (amino acid residues from about 196-319), “Domain III” (amino acid residues from about 320-488), and “Domain IV” (amino acid residues from about 489-630) (residue numbering without signal peptide). See Garrett et al. Mol. Cell. 11: 495-505 (2003), Cho et al. Nature 421: 756-760 (2003), Franklin et al. Cancer Cell 5:317-328 (2004), and Plowman et al. Proc. Natl. Acad. Sci. 90:1746-1750 (1993).

“ErbB3” and “HER3” refer to the receptor polypeptide as disclosed, for example, in U.S. Pat. Nos. 5,183,884 and 5,480,968 as well as Kraus et al. PNAS (USA) 86:9193-9197 (1989).

Herein, “HER3 extracellular domain” or “HER3ECD” refers to a domain of HER3 that is outside of a cell, either anchored to a cell membrane, or in circulation, including fragments thereof. In one embodiment, the extracellular domain of HER3 may comprise four domains: Domain I, Domain II, Domain III, and Domain IV. In one embodiment, the HER3ECD comprises amino acids 1-636 (numbering including signal peptide). In one embodiment, HER3 domain III comprises amino acids 328-532 (numbering including signal peptide.

The terms “ErbB4” and “HER4” herein refer to the receptor polypeptide as disclosed, for example, in EP Pat Appln No 599,274; Plowman et al., Proc. Natl. Acad. Sci. USA, 90:1746-1750 (1993); and Plowman et al., Nature, 366:473-475 (1993), including isoforms thereof, e.g., as disclosed in WO99/19488, published Apr. 22, 1999.

By “HER ligand” is meant a polypeptide which binds to and/or activates a HER receptor. The HER ligand of particular interest herein is a native sequence human HER ligand such as epidermal growth factor (EGF) (Savage et al., J. Biol. Chem. 247:7612-7621 (1972)); transforming growth factor alpha (TGF-α) (Marquardt et al., Science 223:1079-1082 (1984)); amphiregulin also known as schwanoma or keratinocyte autocrine growth factor (Shoyab et al. Science 243:1074-1076 (1989); Kimura et al. Nature 348:257-260 (1990); and Cook et al. Mol. Cell. Biol. 11:2547-2557 (1991)); betacellulin (Shing et al., Science 259:1604-1607 (1993); and Sasada et al. Biochem. Biophys. Res. Commun. 190:1173 (1993)); heparin-binding epidermal growth factor (HB-EGF) (Higashiyama et al., Science 251:936-939 (1991)); epiregulin (Toyoda et al., J. Biol. Chem. 270:7495-7500 (1995); and Komurasaki et al. Oncogene 15:2841-2848 (1997)); neuregulins including neuregulin1 (NRG1), neuregulin-2 (NRG2) (Carraway et al., Nature 387:512-516 (1997)), neuregulin-3 (NRG3) (Zhang et al., Proc. Natl. Acad. Sci. 94:9562-9567 (1997)), and neuregulin-4 (NRG4) (Harari et al. Oncogene 18:2681-89 (1999)); and cripto (CR-1) (Kannan et al. J. Biol. Chem. 272(6):3330-3335 (1997)). HER ligands which bind EGFR include EGF, TGF-α, amphiregulin, betacellulin, HB-EGF and epiregulin. HER ligands which bind HER3 include NRG1 and NRG2. HER ligands capable of binding HER4 include betacellulin, epiregulin, HB-EGF, NRG1, NRG2, NRG3, and NRG4.

The term “NRG” as used herein, refers to any native neuregulin (also known as heregulin (HRG)) from any vertebrate source, including mammals such as primates (e.g. humans) and rodents (e.g., mice and rats), unless otherwise indicated. The term encompasses “full-length,” unprocessed NRG as well as any form of NRG that results from natural processing. The term also encompasses naturally occurring variants of NRG, e.g., splice variants or allelic variants. There are four known forms of NRG: NRG1 (Holmes, W. E. et al., Science 256:1205-1210 (1992)); NRG2 (Caraway, K. L. et al., Nature 387:512-516 (1997)); NRG3 (Zhang, E. et al., Proc Natl Acad Sci USA 94:9562-9567)); and NRG4 (Harari, D. et al., Oncogene 18:2681-2689)). Due to alternative splicing there are two active isoforms of the NRG1 EGF-like domain that are required for receptor binding, referred to as NRG1 alpha (NRG1α) and NRGlbeta (NRGβ). Sequences of exemplary human NRG1s are shown in Genbank Accession No. BK000383 (Falls, D. L., Ex Cell Res, 284:14-30 (2003) and in U.S. Pat. No. 5,367,060. In one embodiment, NRG1 comprises the amino acid sequence of Swiss Prot accession number Q7RTV8 (SEQ ID NO: 9).

“HER dimer” herein is a noncovalently associated dimer comprising at least two HER receptors. Such complexes may form when a cell expressing two or more HER receptors is exposed to an HER ligand and can be isolated by immunoprecipitation and analyzed by SDS-PAGE as described in Sliwkowski et al., J. Biol. Chem., 269(20):14661-14665 (1994), for example. Other proteins, such as a cytokine receptor subunit (e.g. gp130) may be associated with the dimer.

A “HER heterodimer” herein is a noncovalently associated heterodimer comprising at least two different HER receptors, such as EGFR-HER2, EGFR-HER3, EGFR-HER4, HER2-HER3 or HER2-HER4 heterodimers.

A “HER inhibitor” is an agent which interferes with HER activation or function. Examples of HER inhibitors include HER antibodies (e.g. EGFR, HER2, HER3, or HER4 antibodies); EGFR-targeted drugs; small molecule HER antagonists; HER tyrosine kinase inhibitors; HER2 and EGFR dual tyrosine kinase inhibitors such as lapatinib/GW572016; antisense molecules (see, for example, WO2004/87207); and/or agents that bind to, or interfere with function of, downstream signaling molecules, such as MAPK or Akt. In one embodiment, the HER inhibitor is an antibody which binds to a HER receptor. In one embodiment, the HER inhibitor is a HER3 inhibitor. In embodiment, the inhibitor is a bispecific HER inhibitor, such as one which inhibits both HER3 and EGFR, HER3 and HER2, or HER3 and HER4. In one embodiment, the HER inhibitor is a bispecific antibody that is specific for both HER3 and EGFR. One example of such an inhibitor is the bispecific antibody DL11f, also known as MEHD7945A.

A “HER dimerization inhibitor” or “HDI” is an agent which inhibits formation of a HER homodimer or HER heterodimer. Preferably, the HER dimerization inhibitor is an antibody. However, HER dimerization inhibitors also include peptide and non-peptide small molecules, and other chemical entities which inhibit the formation of HER homo- or heterodimers.

An antibody which “inhibits HER dimerization” is an antibody which inhibits, or interferes with, formation of a HER dimer, regardless of the underlying mechanism. In one embodiment, such an antibody binds to HER2 at the heterodimeric binding site thereof. One particular example of a dimerization inhibiting antibody is pertuzumab (Pmab), or MAb 2C4. Other examples of HER dimerization inhibitors include antibodies which bind to EGFR and inhibit dimerization thereof with one or more other HER receptors (for example EGFR monoclonal antibody 806, MAb 806, which binds to activated or “untethered” EGFR; see Johns et al., J. Biol. Chem. 279(29):30375-30384 (2004)); antibodies which bind to HER3 and inhibit dimerization thereof with one or more other HER receptors; antibodies which bind to HER4 and inhibit dimerization thereof with one or more other HER receptors; peptide dimerization inhibitors (U.S. Pat. No. 6,417,168); antisense dimerization inhibitors; etc.

As used herein, “EGFR antagonist” or “EGFR inhibitor” refer to those compounds that specifically bind to EGFR and prevent or reduce its signaling activity, and do not specifically bind to HER2, HER3, or HER4. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBITUX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Sugen); and AG1571 (SU 5271; Sugen).

A “HER antibody” is an antibody that binds to a HER receptor. Optionally, the HER antibody further interferes with HER activation or function. Particular HER2 antibodies include pertuzumab and trastuzumab. Examples of particular EGFR antibodies include cetuximab and panitumumab. Exemplary anti-HER3 antibodies are described in WO2011076683 (Mab205.10.1, Mab205.10.2, Mab205.10.3), U.S. Pat. No. 7,846,440; U.S. Pat. No. 7,705,130 and U.S. Pat. No. 5,968,511.

Patent publications related to HER antibodies include: U.S. Pat. No. 5,677,171, U.S. Pat. No. 5,720,937, U.S. Pat. No. 5,720,954, U.S. Pat. No. 5,725,856, U.S. Pat. No. 5,770,195, U.S. Pat. No. 5,772,997, U.S. Pat. No. 6,165,464, U.S. Pat. No. 6,387,371, U.S. Pat. No. 6,399,063, US2002/0192211A1, U.S. Pat. No. 6,015,567, U.S. Pat. No. 6,333,169, U.S. Pat. No. 4,968,603, U.S. Pat. No. 5,821,337, U.S. Pat. No. 6,054,297, U.S. Pat. No. 6,407,213, U.S. Pat. No. 6,719,971, U.S. Pat. No. 6,800,738, US2004/0236078A1, U.S. Pat. No. 5,648,237, U.S. Pat. No. 6,267,958, U.S. Pat. No. 6,685,940, U.S. Pat. No. 6,821,515, WO98/17797, U.S. Pat. No. 6,333,398, U.S. Pat. No. 6,797,814, U.S. Pat. No. 6,339,142, U.S. Pat. No. 6,417,335, U.S. Pat. No. 6,489,447, WO99/31140, US2003/0147884A1, US2003/0170234A1, US2005/0002928A1, U.S. Pat. No. 6,573,043, US2003/0152987A1, WO99/48527, US2002/0141993A1, WO01/00245, US2003/0086924, US2004/0013667A1, WO00/69460, WO01/00238, WO01/15730, U.S. Pat. No. 6,627,196B1, U.S. Pat. No. 6,632,979B1, WO01/00244, US2002/0090662A1, WO01/89566, US2002/0064785, US2003/0134344, WO 04/24866, US2004/0082047, US2003/0175845A1, WO03/087131, US2003/0228663, WO2004/008099A2, US2004/0106161, WO2004/048525, US2004/0258685A1, U.S. Pat. No. 5,985,553, U.S. Pat. No. 5,747,261, U.S. Pat. No. 4,935,341, U.S. Pat. No. 5,401,638, U.S. Pat. No. 5,604,107, WO 87/07646, WO 89/10412, WO 91/05264, EP 412,116 B1, EP 494,135 B1, U.S. Pat. No. 5,824,311, EP 444,181 B1, EP 1,006,194 A2, US 2002/0155527A1, WO 91/02062, U.S. Pat. No. 5,571,894, U.S. Pat. No. 5,939,531, EP 502,812 B1, WO 93/03741, EP 554,441 B1, EP 656,367 A1, U.S. Pat. No. 5,288,477, U.S. Pat. No. 5,514,554, U.S. Pat. No. 5,587,458, WO 93/12220, WO 93/16185, U.S. Pat. No. 5,877,305, WO 93/21319, WO 93/21232, U.S. Pat. No. 5,856,089, WO 94/22478, U.S. Pat. No. 5,910,486, U.S. Pat. No. 6,028,059, WO 96/07321, U.S. Pat. No. 5,804,396, U.S. Pat. No. 5,846,749, EP 711,565, WO 96/16673, U.S. Pat. No. 5,783,404, U.S. Pat. No. 5,977,322, U.S. Pat. No. 6,512,097, WO 97/00271, U.S. Pat. No. 6,270,765, U.S. Pat. No. 6,395,272, U.S. Pat. No. 5,837,243, WO 96/40789, U.S. Pat. No. 5,783,186, U.S. Pat. No. 6,458,356, WO 97/20858, WO 97/38731, U.S. Pat. No. 6,214,388, U.S. Pat. No. 5,925,519, WO 98/02463, U.S. Pat. No. 5,922,845, WO 98/18489, WO 98/33914, U.S. Pat. No. 5,994,071, WO 98/45479, U.S. Pat. No. 6,358,682 B1, US 2003/0059790, WO 99/55367, WO 01/20033, US 2002/0076695 A1, WO 00/78347, WO 01/09187, WO 01/21192, WO 01/32155, WO 01/53354, WO 01/56604, WO 01/76630, WO02/05791, WO 02/11677, U.S. Pat. No. 6,582,919, US2002/0192652A1, US 2003/0211530A1, WO 02/44413, US 2002/0142328, U.S. Pat. No. 6,602,670 B2, WO 02/45653, WO 02/055106, US 2003/0152572, US 2003/0165840, WO 02/087619, WO 03/006509, WO03/012072, WO 03/028638, US 2003/0068318, WO 03/041736, EP 1,357,132, US 2003/0202973, US 2004/0138160, U.S. Pat. No. 5,705,157, U.S. Pat. No. 6,123,939, EP 616,812 B1, US 2003/0103973, US 2003/0108545, U.S. Pat. No. 6,403,630 B1, WO 00/61145, WO 00/61185, U.S. Pat. No. 6,333,348 B1, WO 01/05425, WO 01/64246, US 2003/0022918, US 2002/0051785 A1, U.S. Pat. No. 6,767,541, WO 01/76586, US 2003/0144252, WO 01/87336, US 2002/0031515 A1, WO 01/87334, WO 02/05791, WO 02/09754, US 2003/0157097, US 2002/0076408, WO 02/055106, WO 02/070008, WO 02/089842, WO 03/86467, WO 2010/108127, and WO 2011/076683.

“HER activation” refers to activation, or phosphorylation, of any one or more HER receptors. Generally, HER activation results in signal transduction (e.g. that caused by an intracellular kinase domain of a HER receptor phosphorylating tyrosine residues in the HER receptor or a substrate polypeptide). HER activation may be mediated by HER ligand binding to a HER dimer comprising the HER receptor of interest. HER ligand binding to a HER dimer may activate a kinase domain of one or more of the HER receptors in the dimer and thereby results in phosphorylation of tyrosine residues in one or more of the HER receptors and/or phosphorylation of tyrosine residues in additional substrate polypeptides(s), such as Akt or MAPK intracellular kinases.

“Phosphorylation” refers to the addition of one or more phosphate group(s) to a protein, such as a HER receptor, or substrate thereof.

A “heterodimeric binding site” on HER2, refers to a region in the extracellular domain of HER2 that contacts, or interfaces with, a region in the extracellular domain of EGFR, HER3 or HER4 upon formation of a dimer therewith. The region is found in Domain II of HER2. Franklin et al. Cancer Cell 5:317-328 (2004).

A HER2 antibody that “binds to a heterodimeric binding site” of HER2, binds to residues in domain II (and optionally also binds to residues in other of the domains of the HER2 extracellular domain, such as domains I and III), and can sterically hinder, at least to some extent, formation of a HER2-EGFR, HER2-HER3, or HER2-HER4 heterodimer Franklin et al. Cancer Cell 5:317-328 (2004) characterize the HER2-pertuzumab crystal structure, deposited with the RCSB Protein Data Bank (ID Code IS78), illustrating an exemplary antibody that binds to the heterodimeric binding site of HER2.

An antibody that “binds to domain II” of HER2 binds to residues in domain II and optionally residues in other domain(s) of HER2, such as domains I and III.

“Isolated,” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step. In some embodiments, the multispecific anti-HER antibody is an isolated antibody.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50 C; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength, and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in a animal model such as that disclosed in Clynes et al. (Proc. Natl. Acad. Sci. USA) 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol 15:203-234 (1997)). FcRs are reviewed in Ravetch and Kinet, Annu. Rev. Immunol 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells, and neutrophils; with PBMCs and NK cells being preferred. The effector cells can be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), can be performed

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets EGFR, ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releasing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

A “subject” is a vertebrate, preferably a mammal, more preferably a human. Mammals include, but are not limited to, humans, non-human higher primates, primates, farm animals (such as cows), sport animals, pets (such as cats, dogs and horses), and laboratory animals (such as mice and rats).

Protein “expression” refers to conversion of the information encoded in a gene into messenger RNA (mRNA) and then to the protein.

Herein, a sample or cell that “expresses” a protein of interest (such as a neuregulin (such as neuregulin1 (NRG1) and/or HER3) is one in which mRNA encoding the protein, or the protein, including fragments thereof, is determined to be present in the sample or cell.

A sample, cell, tumor or cancer which “expresses NRG1 at a level higher than the median level for NRG1 expression” in a cancer type is one in which the level of NRG1 expression is considered a “high NRG1 level” to a skilled person for that type of cancer. This cancer is also considered to “overexpress NRG1”. Generally, such level will be in the range from about 50% to about 100%, relative to NRG1 levels in a population of samples, cells, tumors, or cancers of the same cancer type. For instance the population which is used to arrive at the median expression level may be HNSCC samples generally, or subgroupings thereof, such as chemotherapy-resistant HNSCC cancer, EGFR inhibitor-resistant HNSCC cancer, as well as advanced, refractory or recurrent HNSCC cancer. The examples herein, demonstrate how the median expression level can be determined This may constitute an absolute value of expression. In one embodiment, the level of NRG1 expression considered a high NRG1 level is at the 60^{th} percentile or higher, 70^{th} percentile or higher, 75^{h} percentile or higher, 80^{th} percentile or higher, 85^{th} percentile or higher, 90^{th} percentile or higher, 95^{th} percentile or higher, 97^{th} percentile or higher, 98^{th} percentile or higher, or, 99^{th} percentile or higher. Such values will be quantified in an assay under specified assay conditions, such as qRT-PCR disclosed herein, and most preferably the qRT-PCR assay as in Example 5. In one embodiment, the NRG1 expression level is calculated using the delta Ct method using an average of the expression level of AL-137727 (SEQ ID NO: 10; SEQ ID NO: 11) and VPS33B (SEQ ID NO: 12) as the reference gene.

In certain cancer types, the NRG1 expression is bimodal in a patient population suffering from that cancer type. The bimodal expression profile consists of a group of patients which exhibit high levels of NRG1 expression—the overexpression mode—and a group of patient which exhibit lower NRG1 expression levels—the lack of overexpression mode. In one embodiment, the inflection point between the two modes is used as the value to characterize a cancer type as either being a cancer which “overexpresses NRG1” (the cancer has a NRG1 expression level that is higher than the inflection point) or a cancer which “lacks overexpression of NRG1” (the cancer has a NRG1 expression level that is lower than the inflection point). In one embodiment, a two-component Gaussian mixture distribution is used to estimate the inflection point between overexpression of NRG1 and lack of overexpression of NRG1. In one embodiment, the NRG1 expression level is calculated using AL-137727 (SEQ ID NO: 10; SEQ ID NO: 11) and VPS33B (SEQ ID NO: 12) as the reference genes.

The technique of “polymerase chain reaction” or “PCR” as used herein generally refers to a procedure wherein minute amounts of a specific piece of nucleic acid, RNA and/or DNA, are amplified as described in U.S. Pat. No. 4,683,195 issued 28 Jul. 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real time polymerase chain reaction” or “qRT-PCR” refers to a form of PCR wherein the amount of PCR product is measured at each step in a PCR reaction. This technique has been described in various publications including Cronin et al., Am. J. Pathol. 164(1):35-42 (2004); and Ma et al., Cancer Cell 5:607-616 (2004).

The term “microarray” refers to an ordered arrangement of hybridizable array elements, preferably polynucleotide probes, on a substrate.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced also increases in the proportion of the number of copies made of the particular gene expressed.

A “native sequence” polypeptide is one which has the same amino acid sequence as a polypeptide (e.g., HER receptor or HER ligand) derived from nature, including naturally occurring or allelic variants. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

Herein, a “patient” is a human patient. The patient may be a “cancer patient,” i.e. one who is suffering or at risk for suffering from one or more symptoms of cancer.

A “tumor sample” herein is a sample derived from, or comprising tumor cells from, a patient's tumor. Examples of tumor samples herein include, but are not limited to, tumor biopsies, circulating tumor cells, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples.

A “fixed” tumor sample is one which has been histologically preserved using a fixative.

A “formalin-fixed” tumor sample is one which has been preserved using formaldehyde as the fixative.

An “embedded” tumor sample is one surrounded by a firm and generally hard medium such as paraffin, wax, celloidin, or a resin. Embedding makes possible the cutting of thin sections for microscopic examination or for generation of tissue microarrays (TMAs).

A “paraffin-embedded” tumor sample is one surrounded by a purified mixture of solid hydrocarbons derived from petroleum.

Herein, a “frozen” tumor sample refers to a tumor sample which is, or has been, frozen.

A cancer or biological sample which “displays HER expression, amplification, or activation” is one which, in a diagnostic test, expresses (including overexpresses) a HER receptor, has amplified HER gene, and/or otherwise demonstrates activation or phosphorylation of a HER receptor.

A cancer cell with “HER receptor overexpression or amplification” is one which has significantly higher levels of a HER receptor protein or gene compared to a noncancerous cell of the same tissue type. Such overexpression may be caused by gene amplification or by increased transcription or translation. HER receptor overexpression or amplification may be determined in a diagnostic or prognostic assay by evaluating increased levels of the HER protein present on the surface of a cell (e.g. via an immunohistochemistry assay; IHC). Alternatively, or additionally, one may measure levels of HER-encoding nucleic acid in the cell, e.g. via fluorescent in situ hybridization (FISH; see WO98/45479 published October, 1998), southern blotting, or polymerase chain reaction (PCR) techniques, such as quantitative real time PCR (qRT-PCR). One may also study HER receptor overexpression or amplification by measuring shed antigen (e.g., HER extracellular domain) in a biological fluid such as serum (see, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

Conversely, a cancer which “does not overexpress or amplify HER receptor” is one which does not have higher than normal levels of HER receptor protein or gene compared to a noncancerous cell of the same tissue type. Antibodies that inhibit HER dimerization, such as pertuzumab, may be used to treat cancer which does not overexpress or amplify HER2 receptor.

Herein, an “anti-tumor agent” refers to a drug used to treat cancer. Non-limiting examples of anti-tumor agents herein include chemotherapeutic agents, HER inhibitors, HER dimerization inhibitors, HER antibodies, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxane, dual tyrosine kinase inhibitors, TLK286, EMD-7200, pertuzumab, trastuzumab, erlotinib, and bevacizumab.

An “approved anti-tumor agent” is a drug used to treat cancer which has been accorded marketing approval by a regulatory authority such as the Food and Drug Administration (FDA) or foreign equivalent thereof.

Where a HER3 inhibitor is administered as a “single anti-tumor agent” it is the only anti-tumor agent administered to treat the cancer, i.e. it is not administered in combination with another anti-tumor agent, such as chemotherapy.

By “standard of care” herein is intended the anti-tumor agent or agents that are routinely used to treat a particular form of cancer.

A “growth inhibitory agent” when used herein refers to a compound or composition which inhibits growth of a cell, especially a HER expressing cancer cell either in vitro or in vivo. Thus, the growth inhibitory agent may be one which significantly reduces the percentage of HER expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13.

Examples of “growth inhibitory” antibodies are those which bind to a HER and inhibit the growth of cancer cells expressing the HER.

An antibody which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody which induces apoptosis is one which results in about 2 to 50 fold, preferably about 5 to 50 fold, and most preferably about 10 to 50 fold, induction of annexin binding relative to untreated cell in an annexin binding assay using BT474 cells.

“Treatment” refers to both therapeutic treatment and prophylactic or preventative measures. Those in need of treatment include those already with cancer as well as those in which cancer is to be prevented. Hence, the patient to be treated herein may have been diagnosed as having cancer or may be predisposed or susceptible to cancer.

The terms “therapeutically effective amount” or “effective amount” refer to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete respose, CR), improve survival (including overall survival and progression free survival) and/or improve one or more symptoms of cancer (e.g. as assessed by FOSI). Most preferably, the therapeutically effective amount of the drug is effective to improve progression free survival (PFS) and/or overall survival (OS).

“Survival” refers to the patient remaining alive, and includes overall survival as well as progression free survival.

“Overall survival” refers to the patient remaining alive for a defined period of time, such as 1 year, 5 years, etc from the time of diagnosis or treatment.

“Progression free survival” refers to the patient remaining alive, without the cancer progressing or getting worse.

By “extending survival” is meant increasing overall or progression free survival in a treated patient relative to an untreated patient (i.e. relative to a patient not treated with a HER inhibitor, such as the bispecific HER3/EGFR inhibitor MEHD7945A), or relative to a patient who does not express NRG1 at the designated level, and/or relative to a patient treated with an approved anti-tumor agent.

An “objective response” refers to a measurable response, including complete response (CR) or partial response (PR).

By “complete response” or “CR” is intended the disappearance of all signs of cancer in response to treatment. This does not always mean the cancer has been cured.

“Partial response” or “PR” refers to a decrease in the size of one or more tumors or lesions, or in the extent of cancer in the body, in response to treatment.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, R¹⁸⁶, R¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

By “chemotherapy-resistant” cancer is meant that the cancer patient has progressed while receiving a chemotherapy regimen (i.e. the patient is “chemotherapy refractory”), or the patient has progressed within 12 months (for instance, within 6 months) after completing a chemotherapy regimen.

By “platinum-resistant” cancer is meant that the cancer patient has progressed while receiving platinum-based chemotherapy (i.e. the patient is “platinum refractory”), or the patient has progressed within 12 months (for instance, within 6 months) after completing a platinum-based chemotherapy regimen.

An “anti-angiogenic agent” refers to a compound which blocks, or interferes with to some degree, the development of blood vessels. The anti-angiogenic factor may, for instance, be a small molecule or antibody that binds to a growth factor or growth factor receptor involved in promoting angiogenesis. The preferred anti-angiogenic factor herein is an antibody that binds to vascular endothelial growth factor (VEGF), such as bevacizumab (AVASTIN®).

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

A “tyrosine kinase inhibitor” is a molecule which inhibits tyrosine kinase activity of a tyrosine kinase such as a HER receptor. Examples of such inhibitors include the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; GW572016 (available from Glaxo) an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibits Raf-1 signaling; non-HER targeted TK inhibitors such as Imatinib mesylate (Gleevac™) available from Glaxo; MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); Imatinib mesylate (Gleevac; Novartis); PKI 166 (Novartis); GW2016 (Glaxo SmithKline); CI-1033 (Pfizer); EKB-569 (Wyeth); Semaxinib (Sugen); ZD6474 (AstraZeneca); PTK-787 (Novartis/Schering AG); INC-1C11 (Imclone); or as described in any of the following patent publications: U.S. Pat. No. 5,804,396; WO99/09016 (American Cyanimid); WO98/43960 (American Cyanamid); WO97/38983 (Warner Lambert); WO99/06378 (Warner Lambert); WO99/06396 (Warner Lambert); WO96/30347 (Pfizer, Inc); WO96/33978 (Zeneca); WO96/3397 (Zeneca); and WO96/33980 (Zeneca).

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks.

A “medicament” is an active drug to treat cancer, such as a HER3 inhibitor, such as a bispecific HER3/EGFR inhibitor (such as MEHD7945A).

A “target audience” is a group of people or an institution to whom or to which a particular medicament is being promoted or intended to be promoted, as by marketing or advertising, especially for particular uses, treatments, or indications, such as individual patients, patient populations, readers of newspapers, medical literature, and magazines, television or internet viewers, radio or internet listeners, physicians, drug companies, etc.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

### II. Compositions and Methods

A. Exemplary HER3 Inhibitors

In one aspect, the invention is based, in part, on selecting a therapy for a patient with a type of cancer based on the expression level of NRG1 in the patient's cancer. In one embodiment, a HER3 inhibitor is selected as the therapy for the patient if the patient's cancer overexpresses NRG1. In one embodiment, a HER3 inhibitor is selected as the therapy for the patient if the NRG1 level in the patient's cancer is higher than the NRG1 level in that cancer type in general. The patient is then treated with the HER3 inhibitor if such a therapy is selected, in certain embodiments.

The HER3 inhibitor can be an antibody or other antigen-binding protein, a small molecule, a nucleic acid (such as an siRNA), or any other such molecule. The HER3 inhibitor, in specific embodiments of the invention, inhibits NRG1 binding to HER3.

In one embodiment, the HER3 inhibitor is an antibody. Exemplary anti-HER3 antibodies are described in WO2011076683 (Mab205.10.1, Mab205.10.2, Mab205.10.3), U.S. Pat. No. 7,846,440; U.S. Pat. No. 7,705,130 and U.S. Pat. No. 5,968,511.

In one embodiment, the HER3 inhibitor is a bispecific HER3/EGFR inhibitor. In one embodiment, the bispecific HER3/EGFR inhibitor is a bispecific antibody. In one embodiment, the bispecific HER3/EGFR inhibitor is a bispecific antibody which comprises an antigen binding domain that specifically binds to both HER3 and EGFR. In one embodiment, the bispecific HER3/EGFR inhibitor is a bispecific antibody which comprises two identical antigen binding domains, each of which specifically binds to both HER3 and EGFR. Such antibodies are described in WO2010108127, US20100255010 and Schaefer et al, Cancer Cell, 20: 472-486 (2011). One such particular bispecific HER3/EGFR inhibitor comprising an antigen binding domain that specifically binds to both HER3 and EGFR is MEHD7945A.

In one embodiment, the bispecific HER3/EGFR antibody comprises an antigen-binding domain that specifically binds to HER3 and EGFR where the antibody comprises a V_{H} comprising one, two, and/or three of the HVRs of the amino acid sequence of SEQ ID NO: 1. In one embodiment, the bispecific HER3/EGFR antibody comprises an antigen-binding domain that specifically binds to HER3 and EGFR where the antibody comprises a V_{H} comprising one, two, and/or three of the HVRs of the amino acid sequence of SEQ ID NO: 1 and a V_{L} comprising one, two, and/or three of the HVRs of the amino acid sequence of SEQ ID NO: 2. In one embodiment, the bispecific HER3/EGFR antibody comprises an antigen-binding domain that specifically binds to HER3 and EGFR where the antibody comprises a V_{H} comprising all three HVRs of the amino acid sequence of SEQ ID NO: 1 and a V_{L} comprising all three of the HVRs of the amino acid sequence of SEQ ID NO: 2. In some embodiments, the HVRs are extended HVRs. In one specific embodiment, HVR-H1 comprises the amino acid sequence LSGDWIH (SEQ ID NO: 3), HVR-H2 comprises the amino acid sequence VGEISAAGGYTD (SEQ ID NO: 4), HVR-H3 comprises the amino acid sequence ARESRVSFEAAMDY (SEQ ID NO: 5), HVR-L1 comprises the amino acid sequence NIATDVA (SEQ ID NO: 6), HVR-L2 comprises the amino acid sequence SASF (SEQ ID NO: 7), and HVR-L3 comprises the amino acid sequence SEPEPYT (SEQ ID NO: 8).

In one embodiment, the bispecific HER3/EGFR antibody comprises an antigen-binding domain that specifically binds to HER3 and EGFR where the antibody comprises a V_{H} having at least 80%, 85%, 90%, 91%, 92%, 93%, 94%, 95%, 96%, 97%, 98%, or 99% sequence identity to the amino acid sequence of SEQ ID NO: 1. In one specific embodiment, the bispecific HER3/EGFR comprising a V_{H} having at least 80%, 85%, 90%, 91%, 92%, 93%, 94%, 95%, 96%, 97%, 98%, or 99% sequence identity to the amino acid sequence of SEQ ID NO: 1 comprises a HVR-H1 comprising the amino acid sequence LSGDWIH (SEQ ID NO: 3), HVR-H2 comprising the amino acid sequence VGEISAAGGYTD (SEQ ID NO: 4), and HVR-H3 comprising the amino acid sequence ARESRVSFEAAMDY (SEQ ID NO: 5),

In one embodiment, the bispecific HER3/EGFR antibody comprises an antigen-binding domain that specifically binds to HER3 and EGFR where the antibody comprises a V_{L} having at least 80%, 85%, 90%, 91%, 92%, 93%, 94%, 95%, 96%, 97%, 98%, or 99% sequence identity to the amino acid sequence of SEQ ID NO: 2. In one specific embodiment, the bispecific HER3/EGFR comprising a V_{L} having at least 80%, 85%, 90%, 91%, 92%, 93%, 94%, 95%, 96%, 97%, 98%, or 99% sequence identity to the amino acid sequence of SEQ ID NO: 2 comprises a HVR-L1 comprising the amino acid sequence NIATDVA (SEQ ID NO: 6), HVR-L2 comprising the amino acid sequence SASF (SEQ ID NO: 7), and HVR-L3 comprising the amino acid sequence SEPEPYT (SEQ ID NO: 8).

In one embodiment, the bispecific HER3/EGFR antibody comprises an antigen-binding domain that specifically binds to HER3 and EGFR where the antibody comprises a V_{H} having at least 80%, 85%, 90%, 91%, 92%, 93%, 94%, 95%, 96%, 97%, 98%, or 99% sequence identity to the amino acid sequence of SEQ ID NO: 1 and a V_{L} having at least 80%, 85%, 90%, 91%, 92%, 93%, 94%, 95%, 96%, 97%, 98%, or 99% sequence identity to the amino acid sequence of SEQ ID NO: 2. In one embodiment, the bispecific HER3/EGFR antibody comprising a V_{H} having at least 80%, 85%, 90%, 91%, 92%, 93%, 94%, 95%, 96%, 97%, 98%, or 99% sequence identity to the amino acid sequence of SEQ ID NO: 1 and a V_{L} having at least 80%, 85%, 90%, 91%, 92%, 93%, 94%, 95%, 96%, 97%, 98%, or 99% sequence identity to the amino acid sequence of SEQ ID NO: 2 comprises a HVR-H1 comprising the amino acid sequence LSGDWIH (SEQ ID NO: 3), HVR-H2 comprising the amino acid sequence VGEISAAGGYTD (SEQ ID NO: 4), and HVR-H3 comprising the amino acid sequence ARESRVSFEAAMDY (SEQ ID NO: 5), a HVR-L1 comprising the amino acid sequence NIATDVA (SEQ ID NO: 6), HVR-L2 comprising the amino acid sequence SASF (SEQ ID NO: 7), and HVR-L3 comprising the amino acid sequence SEPEPYT (SEQ ID NO: 8).

In one embodiment, the bispecific HER3/EGFR antibody comprises an antigen-binding domain that specifically binds to HER3 and EGFR where the antibody comprises a V_{H} comprising the amino acid sequence of SEQ ID NO: 1. In one embodiment, the bispecific HER3/EGFR antibody comprises an antigen-binding domain that specifically binds to HER3 and EGFR where the antibody comprises a V_{L} comprising the amino acid sequence of SEQ ID NO: 2. In one embodiment, the bispecific HER3/EGFR antibody comprises an antigen-binding domain that specifically binds HER3 and EGFR where the antibody comprises a V_{H} comprising the amino acid sequence of SEQ ID NO: 1 and a V_{L} comprising the amino acid sequence of SEQ ID NO: 2.

1. Antibody Affinity

In certain embodiments, an antibody provided herein has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μL1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μL1/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB®technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA. 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol, 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g. a traditional bispecific antibody comprising two antigen binding domains each specific for a distinct target. Multispecific antibodies are monoclonal antibodies that have binding specificities for at least two different sites. In certain embodiments, one of the binding specificities is for HER3 and the other is for any other antigen. In certain embodiments, bispecific antibodies may bind to two different epitopes of HER3. Bispecific antibodies may also be used to localize cytotoxic agents to cells which express HER3. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to HER3 as well as another, different antigen (see, US 2008/0069820, for example). Examples of such a bispecific HER3/EGFR inhibitor are described herein and include the exemplary MEHD7945A antibody.

7. Antibody Variants

In certain embodiments, amino acid sequence variants of the antibodies provided herein are contemplated. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: H is, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Recombinant Methods and Compositions

Antibodies may be produced using recombinant methods and compositions, e.g., as described in U.S. Pat. No. 4,816,567. In one embodiment, isolated nucleic acid encoding an anti-HER3 antibody (including bispecific antibodies) described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti HER3 antibody (including bispecific antibodies) is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti HER3 antibody (including bispecific antibodies), nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### III. Diagnostic Methods

One aspect of the invention provides a method for selecting a therapy for a patient with cancer comprising determining neuregulin 1 (NRG1) expression in a cancer sample from the patient and selecting a HER3 inhibitor for therapy if the cancer overexpresses NRG1.

Another aspect of the invention provides a method for selecting a therapy for a patient with cancer comprising determining neuregulin 1 (NRG1) expression in a cancer sample from the patient and selecting a bispecific HER3/EGFR inhibitor as the therapy if the sample overexpresses NRG1.

In one embodiment, the patient's cancer expresses NRG1 at a level higher than the median level for NRG1 expression in the cancer type. In one embodiment, the level of NRG1 expression considered a high NRG1 level is at the 60^{th} percentile or higher, 70^{th} percentile or higher, 75^{h} percentile or higher, 80^{th} percentile or higher, 85^{th} percentile or higher), 90^{th} percentile or higher, 95^{th} percentile or higher, or more (97^{th} percentile or higher), etc. The median or percentile expression level can be determined essentially contemporaneously with measuring NRG1 expression or may have been determined previously.

In certain cancer types, the NRG1 expression is bimodal in a patient population suffering from that cancer type. The bimodal expression profile consists of a group of patients which exhibit high levels of NRG1 expression—the overexpression mode—and a group of patient which exhibit lower NRG1 expression levels—the lack of overexpression mode. In one embodiment, the inflection point between the two modes is used as the value to characterize a cancer type as either being a cancer which overexpresses NRG1 or a cancer which lacks overexpression of NRG1. A cancer which has a NRG1 expression level that is higher than the inflection point would be characterized as a cancer type which overexpresses NRG1. A cancer which has a NRG1 expression level that is lower than the inflection point would be characterized as a cancer type which lacks overexpression of NRG1.

Example 4 provides assays for determining the distribution of NRG1 expression in a patient population. In one embodiment, a two-component Gaussian mixture distribution is used to estimate the inflection point between overexpression of NRG1 and lack of overexpression of NRG1.

One example of a cancer that is shown herein to exhibit a bimodal NRG1 expression profile is head and neck squamous cell carcinoma (HNSCC). As discussed in Example 4, a population of HNSCC cancers exhibits a Gaussian bimodal distribution profile of cancers with NRG1 overexpression and cancers lacking NRG1 overexpression. The inflection point provided by the distribution analysis is around 0.3689 on the logarithm scale, corresponding to approximately 1.50 on the linear scale.

In one embodiment, the cancer which overexpresses NRG1 also exhibits neuregulin-induced autocrine signaling. A cancer which exhibits neuregulin-induced autocrine signaling can be identified by the presence of co-expression of NRG1 and HER3 in the cancer cells. Co-expression of NRG1 and HER3 can be measured, for example, by RNA in situ hybridization procedures.

Also provided herein is a method of quantifying NRG1 expression level in a cancer sample comprising determining the expression level of NRG1 in the sample, and quantifying the expression level of NRG1 in the sample relative to the expression level of one or more reference genes in the sample. Suitable reference genes include AL-137727, VPS33B, GAPDH, SDHA, SP2, GUSB, etc. In one embodiment, the method of quantifying NRG1 expression level in a cancer sample comprises determining the expression level of NRG1 in the sample, and quantifying the expression level of NRG1 in the sample relative to the expression level of one or both of AL-137727 (SEQ ID NO: 10, SEQ ID NO: 11) and VPS33B (SEQ ID NO: 12) in the sample. In one embodiment the expression level of NRG1 in the sample is quantified relative to the expression level of AL-137727. In one embodiment the expression level of NRG1 in the sample is quantified relative to the expression level of VPS33B. In one embodiment the expression level of NRG1 in the sample is quantified relative to the expression level of both of AL-137727 and VPS33B in the sample. In one embodiment, the NRG1 expression level is calculated using the delta Ct method using an average of the expression level of AL-137727 and VPS33B. This method of quantifying NRG1 expression provides a reliable standard for comparing NRG1 expression for the purposes of selecting a therapy for a patient or for predicting the response of a patient to a therapy.

Prior to the therapeutic methods described below, NRG1 expression level(s) in the patient's cancer is/are assessed. Generally, a biological sample is obtained from the patient in need of therapy, which sample is subjected to one or more diagnostic assay(s), usually at least one in vitro diagnostic (IVD) assay. However, other forms of evaluating NRG1 expression, such as in vivo diagnosis, are expressly contemplated herein.

The biological sample is, for example, tumor sample, a blood sample, sputum sample, a urine sample, or other tissue or bodily fluid retrieved from the patient. In some embodiments, the biological sample is a fixed sample, e.g. a formalin fixed, paraffin-embedded (FFPE) sample, or a frozen sample. In certain embodiments, expression level of NRG1 is determined in situ.

Various methods for determining expression of mRNA or protein include, but are not limited to, gene expression profiling, polymerase chain reaction (PCR) including quantitative real time PCR (qRT-PCR), microarray analysis, serial analysis of gene expression (SAGE), MassARRAY, Gene Expression Analysis by Massively Parallel Signature Sequencing (MPSS), proteomics, immunohistochemistry (IHC), direct RNA sequencing, mass spectrometry, ELISAs, etc. Preferably mRNA is quantified. Such mRNA analysis is preferably performed using the technique of polymerase chain reaction (PCR), or by microarray analysis. Where PCR is employed, a preferred form of PCR is quantitative real time PCR (qRT-PCR). The preferred qRT-PCR assay is that as described in Example 1 below.

The steps of a representative protocol for profiling gene expression using fixed, paraffin-embedded tissues as the RNA source, including mRNA isolation, purification, primer extension and amplification are given in various published journal articles (for example: Godfrey et al. J. Molec. Diagnostics 2: 84-91 (2000); Specht et al., Am. J. Pathol. 158: 419-29 (2001)). Briefly, a representative process starts with cutting about 10 microgram thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR. Finally, the data are analyzed to identify the best treatment option(s) available to the patient on the basis of the characteristic gene expression pattern identified in the tumor sample examined.

Various exemplary methods for determining gene expression will now be described in more detail.

(i) Gene Expression Profiling

In general, methods of gene expression profiling can be divided into two large groups: methods based on hybridization analysis of polynucleotides, and methods based on sequencing of polynucleotides. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and polymerase chain reaction (PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS), and direct sequencing of RNA

(ii) Polymerase Chain Reaction (PCR)

Of the techniques listed above, a sensitive and flexible quantitative method is PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, California, USA), following the manufacturer's instructions. The derived cDNA can then be used as a template in the subsequent PCR reaction. Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® PCR can be performed using commercially available equipment, such as, for example, ABI PRISM 7700® Sequence Detection System® (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700® Sequence Detection System. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and P-actin.

A more recent variation of the PCR technique is quantitative real time PCR (qRT-PCR), which measures PCR product accumulation through a dual-labeled fluorigenic probe (i.e., TAQMAN® probe). Real time PCR is compatible both with quantitative competitive PCR, where internal competitor for each target sequence is used for normalization, and with quantitative comparative PCR using a normalization gene contained within the sample, or a housekeeping gene for PCR. For further details see, e.g. Held et al., Genome Research 6:986-994 (1996).

The steps of a representative protocol for profiling gene expression using fixed, paraffin-embedded tissues as the RNA source, including mRNA isolation, purification, primer extension and amplification are given in various published journal articles (for example: Godfrey et al., J. Molec. Diagnostics 2: 84-91 (2000); Specht et al., Am. J. Pathol. 158: 419-29 (2001)). Briefly, a representative process starts with cutting about 10 microgram thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR.

According to one aspect of the present invention, PCR primers and probes are designed based upon intron sequences present in the gene to be amplified. In this embodiment, the first step in the primer/probe design is the delineation of intron sequences within the genes. This can be done by publicly available software, such as the DNA BLAT software developed by Kent, W., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Rozen and Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

Factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach et al., “General Concepts for PCR Primer Design” in PCR Primer, A Laboratory Manual, Cold Spring Harbor Laboratory Press, New York, 1995, pp. 133-155; Innis and Gelfand, “Optimization of PCRs” in PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

The preferred conditions, primers, probes, and internal reference (G6PDH) are as described in Example 1 below.

(iii) Microarrays

Differential gene expression can also be identified, or confirmed using the microarray technique. Thus, the expression profile of breast cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GENCHIP™ technology, or Incyte's microarray technology.

The development of microarray methods for large-scale analysis of gene expression makes it possible to search systematically for molecular markers of cancer classification and outcome prediction in a variety of tumor types.

(iv) Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

(v) MassARRAY Technology

The MassARRAY (Sequenom, San Diego, Calif.) technology is an automated, high-throughput method of gene expression analysis using mass spectrometry (MS) for detection. According to this method, following the isolation of RNA, reverse transcription and PCR amplification, the cDNAs are subjected to primer extension. The cDNA-derived primer extension products are purified, and dipensed on a chip array that is pre-loaded with the components needed for MALTI-TOF MS sample preparation. The various cDNAs present in the reaction are quantitated by analyzing the peak areas in the mass spectrum obtained.

(vi) Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 microgram diameter microbeads. First, a microbead library of DNA templates is constructed by in vitro cloning. This is followed by the assembly of a planar array of the template-containing microbeads in a flow cell at a high density (typically greater than 3×106 microbeads/cm2). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

(vii) Immunohistochemistry

Immunohistochemistry methods are also suitable for detecting the expression levels of the prognostic markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody Immunohistochemistry protocols and kits are well known in the art and are commercially available.

(viii) Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

(ix) Direct RNA Sequencing

Direct RNA Sequencing (DSR) allows massively parallel sequencing of RNA molecules directly without prior synthesis of cDNA or the need for ligation/amplification steps. Ozsolak, et al., Nature 461:814-818 (2009); Ozsolak F, and Milos P M., WIREs RNA, 2: 565-570 (2011), Ozsolak F, and Milos P M., Experimental Medicine 28: 2574-2580. This technique allows for quantification and characterization of RNA samples, including those formalin-fixed and paraffin embedded.

In general, isolated total RNA or cell lysates are added to tpoly(dT)-coated flow cells, which enable capture and sequencing of polyA RNA species. PolyA polymerase is used to generate a polyA tail before loading the sample to the flow cells for sequencing for RNA species which do not contain a natural polyA tail.

(x) RNA In Situ Hybridization

RNA in situ hybridization (RISH) is technique used for the examination of mRNA expression in tissue samples. Veeck J and Dahl E., Methods Mol. Biol., 664:135-50 (2010); Nuovo G J., Methods, 44:39-46 (2008); Yamada H., Cytometry A., 77:1032-7 2010.

In general, a probe specific for the RNA of interest is labeled with a detectable label, such as a radioactive tag, an enzymatic probe, a chemical dye or fluorescent compound. A tissue sample of interest is contacted with a solution of single-stranded labeled probe under conditions that allow the probe to hybridize to complementary RNA sequences in the cells. Any unhybridized probe is removed and the hybridized probe is detected by the appropriated method.

(xi) General Description of mRNA Isolation, Purification and Amplification

The steps of a representative protocol for profiling gene expression using fixed, paraffin-embedded tissues as the RNA source, including mRNA isolation, purification, primer extension and amplification are given in various published journal articles (for example: Godfrey et al. J. Molec. Diagnostics 2: 84-91 (2000); Specht et al., Am. J. Pathol. 158: 419-29 (2001)). Briefly, a representative process starts with cutting about 10 microgram thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR. Finally, the data are analyzed to identify the best treatment option(s) available to the patient on the basis of the characteristic gene expression pattern identified in the tumor sample examined.

NRG1 expression may also be evaluated using an in vivo diagnostic assay, e.g. by administering a molecule (such as an antibody) which binds the molecule to be detected and is tagged with a detectable label (e.g. a radioactive isotope) and externally scanning the patient for localization of the label.

### IV. Pharmaceutical Formulations

Therapeutic formulations of the HER3 inhibitor, such as a bispecific HER3/EGFR inhibitor, used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), generally in the form of lyophilized formulations or aqueous solutions. Antibody crystals are also contemplated (see US Pat Appln 2002/0136719). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized antibody formulations are described in WO 97/04801, expressly incorporated herein by reference.

The formulation herein may also contain more than one active compound as necessary for the particular indication being treated, preferably those with complementary activities that do not adversely affect each other. Various drugs which can be combined with the HER3 inhibitor are described in the Treatment Section below. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

Accordingly, a method for manufacturing a HER3 inhibitor, such as a bispecific HER3/EGFR inhibitor, (such as MEHD7945A), or a pharmaceutical composition thereof is provided, which method comprises combining in a package the inhibitor or pharmaceutical composition and a label stating that the inhibitor or pharmaceutical composition is indicated for treating a patient with a type of cancer (for example, HNSCC) which is able to respond to the inhibitor, wherein the patient's cancer expresses NRG1 at a level higher than the median level for NRG1 expression in the cancer type.

In addition, a method for manufacturing a chemotherapeutic agent or a pharmaceutical composition thereof is provided, wherein the method comprises combining in a package the chemotherapeutic agent or pharmaceutical composition and a label stating that the chemotherapeutic agent or pharmaceutical composition is indicated for treating a patient with a type of cancer, wherein the patient's cancer expresses NRG1 at a level higher than the median level for NRG1 expression in the cancer type.

### V. Treatment with HER3 Inhibitors

The invention herein provides methods of treating a cancer patient comprising administering a therapeutically effective amount of a HER3 inhibitor, such as a bispecific HER3/EGFR inhibitor, to the patient, wherein the patient, prior to administration of the HER3 inhibitor, was diagnosed with a type of cancer which overexpresses NRG1.

In one embodiment, the diagnosis of overexpression of NRG1 was based on the determination that the patient's cancer expresses NRG1 at a level higher than the median level for NRG1 expression in the cancer type. In a certain embodiment, the patient's cancer expresses NRG1 at a level which is at or higher than the 60^{th}, 65^{th}, 70^{th}, 75^{th}, 80th, 85^{th}, 90^{th}, 95^{th}, 97^{th} percentile for NRG1 expression in the cancer type.

In certain cancer types, the NRG1 expression is bimodal in a patient population suffering from that cancer type. The bimodal expression profile consists of a group of patients which exhibit high levels of NRG1 expression—the overexpression mode—and a group of patient which exhibit lower NRG1 expression levels—the lack of overexpression mode. In one embodiment, the inflection point between the two modes is used as the value to characterize a cancer type as either being a cancer which overexpresses NRG1 or a cancer which lacks overexpression of NRG1. A cancer which has a NRG1 expression level that is higher than the inflection point would be characterized as a cancer type which overexpresses NRG1. A cancer which has a NRG1 expression level that is lower than the inflection point would be characterized as a cancer type which lacks overexpression of NRG1. Accordingly, in one embodiment, the diagnosis of overexpression of NRG1 was based on the determination that the patient's cancer falls into the overexpression mode of a bimodal NRG1 expression profile.

Example 4 provides assays for determining the distribution of NRG1 expression in a patient population. In one embodiment, a two-component Gaussian mixture distribution is used to estimate the inflection point between overexpression of NRG1 and lack of overexpression of NRG1.

In one embodiment, the cancer to be treated exhibits a bimodal NRG1 expression profile. One example of such a cancer is head and neck squamous cell carcinoma (HNSCC). As discussed in Example 4, a population of HNSCC cancers exhibits a Gaussian bimodal distribution profile of cancers with NRG1 overexpression and cancers lacking NRG1 overexpression. The inflection point provided by the distribution analysis is around 0.3689 on the logarithm scale, corresponding to approximately 1.50 on the linear scale.

In one embodiment, the cancer to be treated exhibits neuregulin-induced autocrine signaling. Such a cancer can be identified by the presence of co-expression of NRG1 and HER3 in the cancer cells. Co-expression of NRG1 and HER3 can be measured, for example, by RNA in situ hybridization procedures.

In one embodiment, the invention provides a method for treating a patient with cancer comprising administering a therapeutically effective amount of MEHD7945A to the patient, wherein the patient, prior to administration of the MEHD7945A, was diagnosed with a type of cancer which overexpresses NRG1. In one embodiment, the cancer exhibits neuregulin-induced autocrine signaling. In one embodiment, the cancer exhibits a bimodal NRG1 expression profile.

In one embodiment, the invention provides a method for treating a patient with a squamous cell carcinoma comprising administering a therapeutically effective amount of MEHD7945A to the patient wherein the patient, prior to administration of the MEHD7945A, was diagnosed with a type of cancer which overexpresses NRG1. In one embodiment, the squamous cell carcinoma exhibits neuregulin-induced autocrine signaling. In one embodiment, the squamous cell carcinoma exhibits a bimodal NRG1 expression profile.

In one embodiment, the invention provides a method for treating a patient with HNSCC comprising administering a therapeutically effective amount of MEHD7945A to the patient wherein the patient, prior to administration of the MEHD7945A, was diagnosed with a type of HNSCC which overexpresses NRG1.

Therapy with the HER3 inhibitor, such as a bispecific HER3/EGFR inhibitor, preferably extends survival, including progression free survival (PFS) and/or overall survival (OS). In one embodiment, therapy with the HER3 inhibitor, such as a bispecific HER3/EGFR inhibitor, extends survival at least about 20% more than survival achieved by administering an approved anti-tumor agent, or standard of care, for the cancer being treated.

The patient may have advanced, refractory, recurrent, chemotherapy-resistant, and/or EGFR inhibitor-resistant cancer. Administration of MEHD7945A the HER3 inhibitor to the patient may, for example, extend survival at least about 20% more than survival achieved by administering an EGFR inhibitor therapy to such a patient.

The HER3 inhibitor, such as a bispecific HER3/EGFR inhibitor is administered to a human patient in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous administration of the antibody is preferred.

For the prevention or treatment of cancer, the dose of the HER3 inhibitor, such as a bispecific HER3/EGFR inhibitor, will depend on the type of cancer to be treated, as defined above, the severity and course of the cancer, whether the antibody is administered for preventive or therapeutic purposes, previous therapy, the patient's clinical history and response to the drug, and the discretion of the attending physician.

In one embodiment, a fixed dose of inhibitor is administered. The fixed dose may suitably be administered to the patient at one time or over a series of treatments. Where a fixed dose is administered, preferably it is in the range from about 20 mg to about 2000 mg of the inhibitor. For example, the fixed dose may be approximately 420 mg, approximately 525 mg, approximately 840 mg, or approximately 1050 mg of the inhibitor.

Where a series of doses are administered, these may, for example, be administered approximately every week, approximately every 2 weeks, approximately every 3 weeks, or approximately every 4 weeks, but preferably approximately every 3 weeks. The fixed doses may, for example, continue to be administered until disease progression, adverse event, or other time as determined by the physician. For example, from about two, three, or four, up to about 17 or more fixed doses may be administered.

In one embodiment, one or more loading dose(s) of the antibody are administered, followed by one or more maintenance dose(s) of the antibody. In another embodiment, a plurality of the same dose are administered to the patient.

While the HER3 inhibitor, such as a bispecific HER3/EGFR inhibitor, may be administered as a single anti-tumor agent, the patient is optionally treated with a combination of the inhibitor (or chemotherapeutic agent), and one or more (additional) chemotherapeutic agent(s). Exemplary chemotherapeutic agents herein include: irinotecan, gemcitabine, carboplatin, paclitaxel, docetaxel, topotecan, and/or liposomal doxorubicin. The combined administration includes coadministration or concurrent administration, using separate formulations or a single pharmaceutical formulation, and consecutive administration in either order, wherein preferably there is a time period while both (or all) active agents simultaneously exert their biological activities. Thus, the antimetabolite chemotherapeutic agent may be administered prior to, or following, administration of the inhibitor. In this embodiment, the timing between at least one administration of the antimetabolite chemotherapeutic agent and at least one administration of the inhibitor is preferably approximately 1 month or less, and most preferably approximately 2 weeks or less. Alternatively, the antimetabolite chemotherapeutic agent and the inhibitor are administered concurrently to the patient, in a single formulation or separate formulations. Treatment with the combination of the chemotherapeutic agent and the inhibitor may result in a synergistic, or greater than additive, therapeutic benefit to the patient.

An antimetabolite chemotherapeutic agent, if administered, is usually administered at dosages known therefor, or optionally lowered due to combined action of the drugs or negative side effects attributable to administration of the antimetabolite chemotherapeutic agent. Preparation and dosing schedules for such chemotherapeutic agents may be used according to manufacturers' instructions or as determined empirically by the skilled practitioner. Where the antimetabolite chemotherapeutic agent is gemcitabine, preferably, it is administered at a dose between about 600 mg/m² to 1250 mg/m² (for example approximately 1000 mg/m²), for instance, on days 1 and 8 of a 3-week cycle.

Aside from the inhibitor and antimetabolite chemotherapeutic agent, other therapeutic regimens may be combined therewith. For example, a second (third, fourth, etc) chemotherapeutic agent(s) may be administered, wherein the second chemotherapeutic agent is either another, different antimetabolite chemotherapeutic agent, or a chemotherapeutic agent that is not an antimetabolite. For example, the second chemotherapeutic agent may be a taxane (such as paclitaxel or docetaxel), capecitabine, or platinum-based chemotherapeutic agent (such as carboplatin, cisplatin, or oxaliplatin), anthracycline (such as doxorubicin, including, liposomal doxorubicin), topotecan, pemetrexed, vinca alkaloid (such as vinorelbine), and TLK 286. “Cocktails” of different chemotherapeutic agents may be administered.

Other therapeutic agents that may be combined with the inhibitor and/or chemotherapeutic agent include any one or more of: a second, different HER inhibitor, HER dimerization inhibitor (for example, a growth inhibitory HER2 antibody such as trastuzumab, or a HER2 antibody which induces apoptosis of a HER2-overexpressing cell, such as 7C2, 7F3 or humanized variants thereof); an antibody directed against a different tumor associated antigen, such as EGFR, HER3, HER4; anti-hormonal compound, e.g., an anti-estrogen compound such as tamoxifen, or an aromatase inhibitor; a cardioprotectant (to prevent or reduce any myocardial dysfunction associated with the therapy); a cytokine; an EGFR-targeted drug (such as TARCEVA® IRESSA® or cetuximab); an anti-angiogenic agent (especially bevacizumab sold by Genentech under the trademark AVASTIN™); a tyrosine kinase inhibitor; a COX inhibitor (for instance a COX-1 or COX-2 inhibitor); non-steroidal anti-inflammatory drug, celecoxib (CELEBREX®); farnesyl transferase inhibitor (for example, Tipifarnib/ZARNESTRA® R115777 available from Johnson and Johnson or Lonafarnib SCH66336 available from Schering-Plough); antibody that binds oncofetal protein CA 125 such as Oregovomab (MoAb B43.13); HER2 vaccine (such as HER2AutoVac vaccine from Pharmexia, or APC8024 protein vaccine from Dendreon, or HER2 peptide vaccine from GSK/Corixa); another HER targeting therapy (e.g. trastuzumab, cetuximab, ABX-EGF, EMD7200, gefitinib, erlotinib, CP724714, CI1033, GW572016, IMC-11F8, TAK165, etc); Raf and/or ras inhibitor (see, for example, WO 2003/86467); doxorubicin HCl liposome injection (DOXIL®); topoisomerase I inhibitor such as topotecan; taxane; HER2 and EGFR dual tyrosine kinase inhibitor such as lapatinib/GW572016; TLK286 (TELCYTA®); EMD-7200; a medicament that treats nausea such as a serotonin antagonist, steroid, or benzodiazepine; a medicament that prevents or treats skin rash or standard acne therapies, including topical or oral antibiotic; a medicament that treats or prevents diarrhea; a body temperature-reducing medicament such as acetaminophen, diphenhydramine, or meperidine; hematopoietic growth factor, etc.

Suitable dosages for any of the above coadministered agents are those presently used and may be lowered due to the combined action (synergy) of the agent and inhibitor.

In addition to the above therapeutic regimes, the patient may be subjected to surgical removal of cancer cells and/or radiation therapy.

Where the inhibitor is an antibody, preferably the administered antibody is a naked antibody. However, the inhibitor administered may be conjugated with a cytotoxic agent. Preferably, the conjugated inhibitor and/or antigen to which it is bound is/are internalized by the cell, resulting in increased therapeutic efficacy of the conjugate in killing the cancer cell to which it binds. In a preferred embodiment, the cytotoxic agent targets or interferes with nucleic acid in the cancer cell. Examples of such cytotoxic agents include maytansinoids, calicheamicins, ribonucleases and DNA endonucleases.

The present application contemplates administration of the inhibitor by gene therapy. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. USA 87:3410-3414 (1990). For review of the currently known gene marking and gene therapy protocols see Anderson et al., Science 256:808-813 (1992). See also WO 93/25673 and the references cited therein.

### VI. Articles of Manufacture

In another embodiment of the invention, an article of manufacture containing materials useful for the treatment of the diseases or conditions described above is provided. The article of manufacture comprises a container and a label or package insert on or associated with the container. Suitable containers include, for example, bottles, vials, syringes, etc. The containers may be formed from a variety of materials such as glass or plastic. The container holds or contains a composition which is effective for treating the disease or condition of choice and may have a sterile access port (for example the container may be an intravenous solution bag or a vial having a stopper pierceable by a hypodermic injection needle). At least one active agent in the composition is the HER3 inhibitor, such as a bispecific HER3/EGFR inhibitor, such as MEHD7945A.

The article of manufacture may further comprise a second container comprising a pharmaceutically-acceptable diluent buffer, such as bacteriostatic water for injection (BWFI), phosphate-buffered saline, Ringer's solution and dextrose solution. The article of manufacture may further include other materials desirable from a commercial and user standpoint, including other buffers, diluents, filters, needles, and syringes.

The kits and articles of manufacture of the present invention also include information, for example in the form of a package insert or label, indicating that the composition is used for treating cancer where the patient's cancer expresses NRG1 at a defined level depending on the drug. The insert or label may take any form, such as paper or on electronic media such as a magnetically recorded medium (e.g., floppy disk) or a CD-ROM. The label or insert may also include other information concerning the pharmaceutical compositions and dosage forms in the kit or article of manufacture.

Generally, such information aids patients and physicians in using the enclosed pharmaceutical compositions and dosage forms effectively and safely. For example, the following information regarding the HER3 inhibitor may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

In a specific embodiment of the invention, an article of manufacture is provided comprising, packaged together, a pharmaceutical composition comprising a HER3 inhibitor, such as a bispecific HER3/EGFR inhibitor, in a pharmaceutically acceptable carrier and a label stating that the inhibitor or pharmaceutical composition is indicated for treating a patient with a type of cancer which is able to respond to a HER3 inhibitor, such as a bispecific HER3/EGFR inhibitor, wherein the patient's cancer expresses NRG1 at a level higher than the median level for NRG1 expression in the cancer type.

In an optional embodiment of this inventive aspect, the article of manufacture herein further comprises a container comprising a second medicament, wherein the HER3 inhibitor is a first medicament, and which article further comprises instructions on the package insert for treating the patient with the second medicament, in an effective amount. The second medicament may be any of those set forth above, with an exemplary second medicament being another HER antibody or a chemotherapeutic agent.

The package insert is on or associated with the container. Suitable containers include, for example, bottles, vials, syringes, etc. The containers may be formed from a variety of materials such as glass or plastic. The container holds or contains a composition that is effective for treating cancer type may have a sterile access port (for example the container may be an intravenous solution bag or a vial having a stopper pierceable by a hypodermic injection needle). At least one active agent in the composition is the HER inhibitor. The label or package insert indicates that the composition is used for treating cancer in a subject eligible for treatment with specific guidance regarding dosing amounts and intervals of inhibitor and any other medicament being provided. The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

Many alternative experimental methods known in the art may be successfully substituted for those specifically described herein in the practice of this invention, as for example described in many of the excellent manuals and textbooks available in the areas of technology relevant to this invention (e.g. Using Antibodies, A Laboratory Manual, edited by Harlow, E. and Lane, D., 1999, Cold Spring Harbor Laboratory Press, (e.g. ISBN 0-87969-544-7); Roe B. A. et. al. 1996, DNA Isolation and Sequencing (Essential Techniques Series), John Wiley & Sons. (e.g. ISBN 0-471-97324-0); Methods in Enzymology: Chimeric Genes and Proteins, 2000, ed. J. Abelson, M. Simon, S. Emr, J. Thomer. Academic Press; Molecular Cloning: a Laboratory Manual, 2001, 3rd Edition, by Joseph Sambrook and Peter MacCallum, (the former Maniatis Cloning manual) (e.g. ISBN 0-87969-577-3); Current Protocols in Molecular Biology, Ed. Fred M. Ausubel, et. al. John Wiley & Sons (e.g. ISBN 0-471-50338-X); Current Protocols in Protein Science, Ed. John E. Coligan, John Wiley & Sons (e.g. ISBN 0-471-11184-8); and Methods in Enzymology: Guide to protein Purification, 1990, Vol. 182, Ed. Deutscher, M. P., Acedemic Press, Inc. (e.g. ISBN 0-12-213585-7)), or as described in the many university and commercial websites devoted to describing experimental methods in molecular biology.

### VII. Methods of Advertising

The invention herein also encompasses a method for advertising a HER3 inhibitor, such as a bispecific HER3/EGFR inhibitor (for instance MEHD7945A) or a pharmaceutically acceptable composition thereof comprising promoting, to a target audience, the use of the inhibitor or pharmaceutical composition thereof for treating a patient population with a type of cancer (such as HNSCC), where the patient's cancer overexpresses NRG1.

Advertising is generally paid communication through a non-personal medium in which the sponsor is identified and the message is controlled. Advertising for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The advertising and promotion of the diagnostic method herein may be accomplished by any means. Examples of advertising media used to deliver these messages include television, radio, movies, magazines, newspapers, the internet, and billboards, including commercials, which are messages appearing in the broadcast media. Advertisements also include those on the seats of grocery carts, on the walls of an airport walkway, and on the sides of buses, or heard in telephone hold messages or in-store PA systems, or anywhere a visual or audible communication can be placed.

More specific examples of promotion or advertising means include television, radio, movies, the internet such as webcasts and webinars, interactive computer networks intended to reach simultaneous users, fixed or electronic billboards and other public signs, posters, traditional or electronic literature such as magazines and newspapers, other media outlets, presentations or individual contacts by, e.g., e-mail, phone, instant message, postal, courier, mass, or carrier mail, in-person visits, etc.

The type of advertising used will depend on many factors, for example, on the nature of the target audience to be reached, e.g., hospitals, insurance companies, clinics, doctors, nurses, and patients, as well as cost considerations and the relevant jurisdictional laws and regulations governing advertising of medicaments and diagnostics. The advertising may be individualized or customized based on user characterizations defined by service interaction and/or other data such as user demographics and geographical location.

Further details of the invention are illustrated by the following non-limiting Examples. The disclosures of all citations in the specification are expressly incorporated herein by reference.

### Example 1

### MEHD7945A is Specific for Both HER3 and EGFR

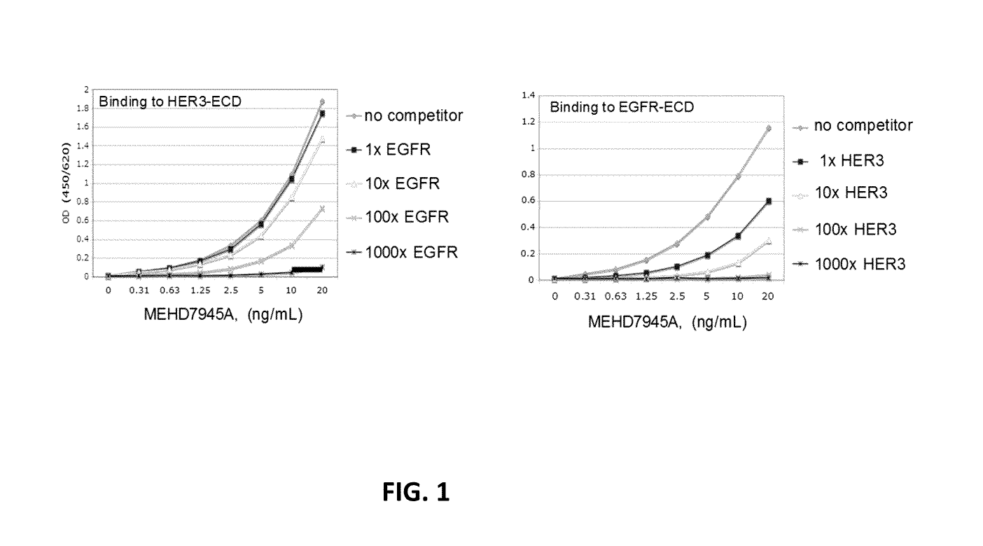

MEHD7945A (also known as DL11f) is an antibody comprising an antigen-binding domain that has binding specificity for both EGFR and HER3. WO 2010/108127 and Schaefer, et al. Cancer Cell, 20: 472-486 (2011). Typically, bitargeting agents are constructed by linking two distinct antigen-binding modules, each module being able to bind to only one antigen. In contrast, in MEHD7945A, each module (Fab) can bind either of two antigens, thus having the potential to elicit enhanced binding affinity from an avidity effect. To confirm that each of the two identical Fabs of MEHD7945A can bind either EGFR or HER3, a competitive binding assay was performed. MEHD7945A binding to immobilized HER3-ECD was reduced in a dose-dependent manner with increasing amounts of EGFR-ECD. Conversely, MEHD7945A was competed from immobilized EGFR-ECD by soluble HER3-ECD protein. As expected, given their relative binding constants, higher concentrations of soluble EGFR-ECD were needed to compete with binding of MEHD7945A to immobilized HER3-ECD (FIG. 1). The results in FIG. 1 are expressed as MEHD7945A concentration versus OD. The assays examined the binding of MEHD7945A to immobilized HER3-ECD or EGFR-ECD, as indicated, in the presence of indicated soluble competitor: 1×=0.02 μg/ml, 10×=0.2 μg/ml, 100×=2 μg/ml, 1000×=20 μg/ml. Results in FIG. 1 are expressed as MEHD7945A concentration versus OD.

### Example 2

### MEHD7945A Inhibits EGFR and HER2/HER3-Dependent Signaling

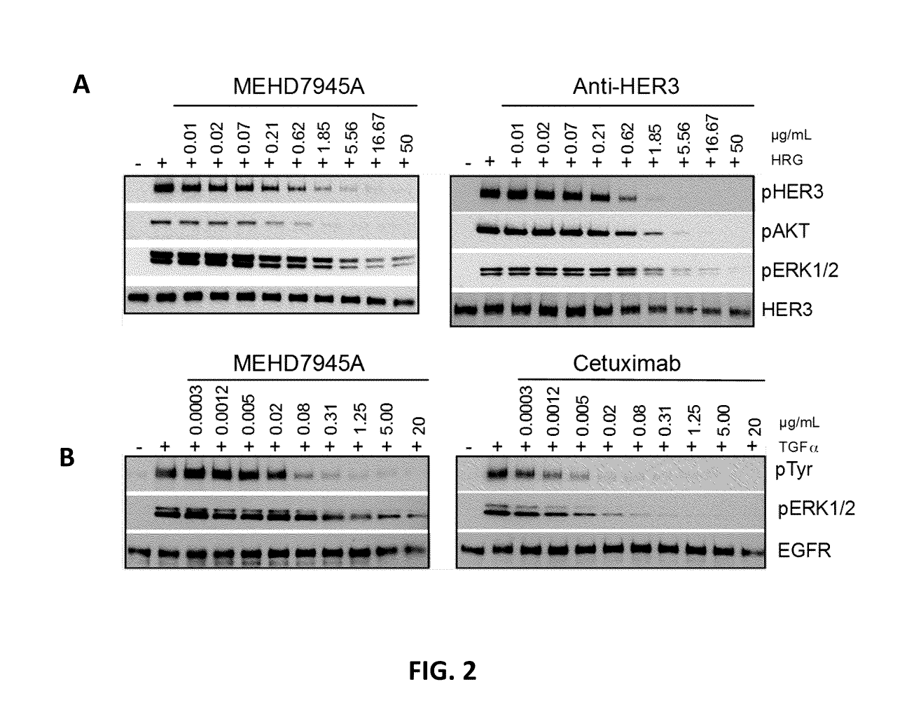

The dual activity of MEHD7945A in cell signaling assays was determined To assess the inhibitory function on HER3, MCF-7 cells for which NRG treatment potently activates the HER2/HER3 pathway were used. Treatment with MEHD7945A prior to NRG stimulation potently inhibited the phosphorylation of HER3 in a dose-dependent manner, and markedly decreased the phosphorylation of AKT and ERK1/2 (FIG. 2A). MEHD7945A inhibited phosphorylation of HER3 with an 1050 of 0.05 μg/ml, phosphorylation of AKT with an 1050 value of 0.19 μg/ml, and phosphorylation of ERK1/2 with an 1050 value of 1.13 μg/ml. Treatment with a monospecific antibody against HER3, anti-HER3, that has comparable binding affinity to HER3 achieved similar results. Anti-HER3 inhibited phosphorylation of HER3 with an 1050 of 0.12 μg/ml, phosphorylation of AKT with an 1050 value of 0.74 μg/ml, and phosphorylation of ERK1/2 with an 1050 value of 1.83 μg/ml. EGFR-NR6 cells were pretreated with MEHD7945A prior to ligand stimulation and it was determined that MEHD7945A inhibited phosphorylation of EGFR and ERK1/2 with 1050 values of 0.03 and 0.16 μg/ml, respectively (FIG. 2B). The monospecific EGFR antibody cetuximab was more effective in inhibiting phosphorylation of EGFR and downstream signaling molecules, which was likely due to the higher binding affinity to EGFR Moreover, betacellulin- and amphiregulin-induced EGFR phosphorylation was also inhibited by MEHD7945A. MEHD7945A inhibited ERK1/2 and AKT pathways as potently as the combination of anti-HER3 and cetuximab in A431 and BxPC3 cells.

The assays were performed as follows. MCF-7 cells treated with indicated concentrations of MEHD7945A or anti-HER3 were stimulated with 0.5 nM NRG for 10 min. Cell lysates were immunoblotted to detect pHER3 (Tyr1289), pAKT (Ser473), pERK1/2 (Thr202/Tyr204), and total HER3. FIG. 2A. EGFR-NR6 cells treated with indicated concentrations of MEHD7945A or cetuximab for 1 hr prior stimulation with 5 nM TGF-α for 10 min. Cell lysates were subjected to immunoblotting to detect, pERK1/2 (Thr202/Tyr204), total EGFR, and phosphorylated EGFR. Since EGFR-NR6 cells only express EGFR all potential phosphorylation sites of EGFR were detected using a pTyr antibody.

### FIG. 2 B

### Example 3

### MEHD7945A is Active in Numerous Cancer Models

### In Vivo Activity in Fadu Xenograft Model, a Head and Neck Squamous Cell Carcinoma Model

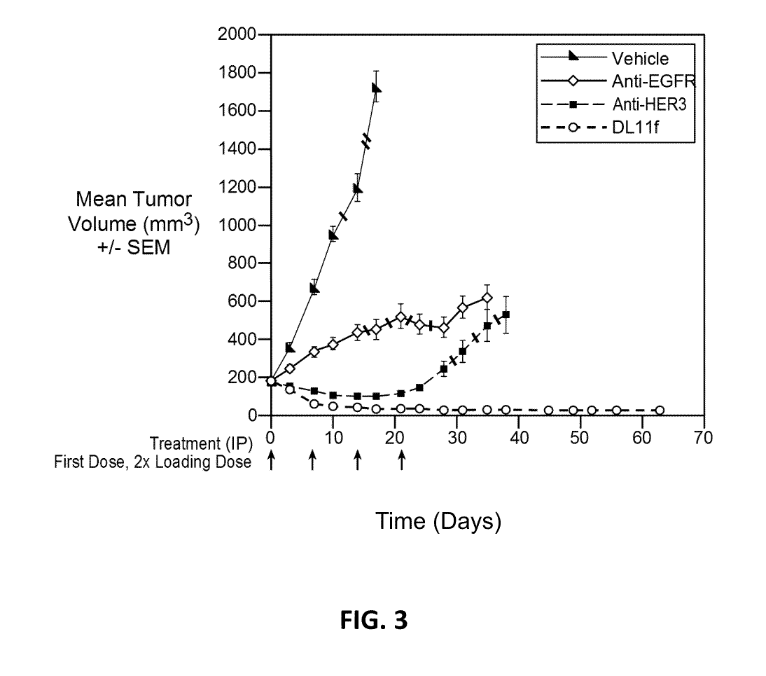

MEHD7945A, a commercially available anti-EGFR antibody, and an anti-HER3 antibody were tested in mice with established tumors derived from Fadu cells (ATCC HTB-43, Manassas, Va.) 5×10⁶ FaDu cells were inoculated subcutaneously in CB17 SCID mice. Animals with similarly sized tumors were randomized into treatment cohorts (n=9/group) as follows: Vehicle (MEHD7945A formulation buffer), anti-EGFR antibody (25 mg/kg), anti-HER3 antibody (50 mg/kg), and MEHD7945A (25 mg/kg). Treatments were administered intraperitoneally, beginning with a 2× loading dose (50 or 100 mg/kg respectively) on the day of randomization and continuing weekly for a total of four treatments. As shown in FIG. 3, MEHD7945A is active in the FaDu head and neck cancer model and is more effective in inhibiting tumor growth than either an anti-EGFR specific or an anti-HER3 specific antibody.

### MEHD7945A is Active in Additional Cancer Types

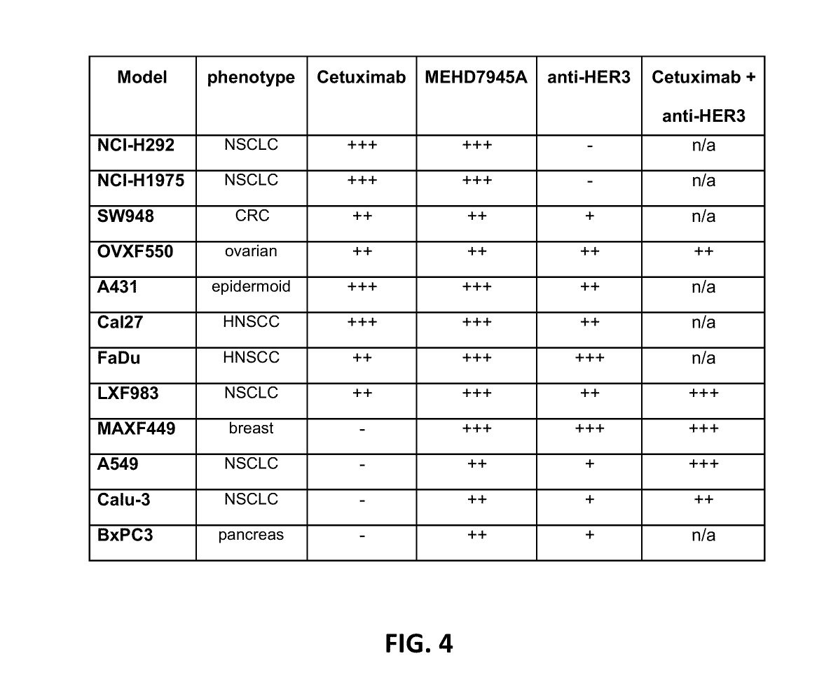

FIG. 4 provides a summary of the some of the additional cancer types in which MEHD7945A shows activity as well as the relative activity of cetuximab or a monospecific anti-HER3 antibody on the cancer types. Details of the assays used to generate this summary are provided in WO 2010/108127. In brief, mice were treated with 25 mg/kg MEHD7945A, 25 mg/kg cetuximab, 50 mg/kg anti-HER3 or the combination of 25 mg/kg cetuximab plus 50 mg/kg anti-HER3, once a week for 4 cycles. MAXF449, OVXF550 and LX983 were treated with 30 mg/kg MEHD7945A, 30 mg/kg cetuximab, 60 mg/kg anti-HER3 or the combination of 30 mg/kg cetuximab plus 60 mg/kg anti-HER3, once a week for 4 cycles. Initial dose was a 2× loading dose for all treatments. Percent of tumor growth inhibition (TGI) was calculated for each study based on the last day of study in which the majority of mice remained in the vehicle group. TGI below 25% is indicated as −, TGI between 25-50% is indicated as +, TGI between 51-75% is indicated as ++, and TGI of 76% and above as +++. NSCLC=non-small cell lung cancer, HNSSC=head and neck squamous cell carcinoma, CRC=colorectal cancer, n/a=non applicable. OVXF550, MAXF449 and LXF983 models are human patient derived transplant models.

### Example 4

### HNSCC Tumors Exhibit Bimodal Expression of NRG1

### Materials and Methods

Immunoprecipitations and Immunoblotting: For immunoprecipitation of tumor tissue, tumor content was verified by H&E requiring a minimum of 50% tumor (most samples had >75% tumor tissue). Tumors were minced on dry ice, then homogenized in chilled lysis buffer (BioWorld, 22040045-2) supplemented with phosphatase and protease inhibitors (Sigma, P5726-5ML, Roche, 13146100). 25 ul anti-HER3 antibody (Santa Cruz, sc-285-G) and 15 ul Dynabeads (Invitrogen, 100.07D) were added to 1-2 mg soluble protein per immunoprecipitation. Samples were then allowed to rotate overnight at 4° C. Beads were then separated using a magnet, and washed three times in lysis buffer. Protein was eluted in sample buffer (Invitrogen, NP0007 and NP0009) by boiling at 95° C. for 5 minutes. Western blotting was performed using standard protocols. 15 ml of eluted protein was loaded per sample and immunodetection was performed using pHER3 (Y1289; #4791) or pan p-Tyr (Clone PY20, EMD).

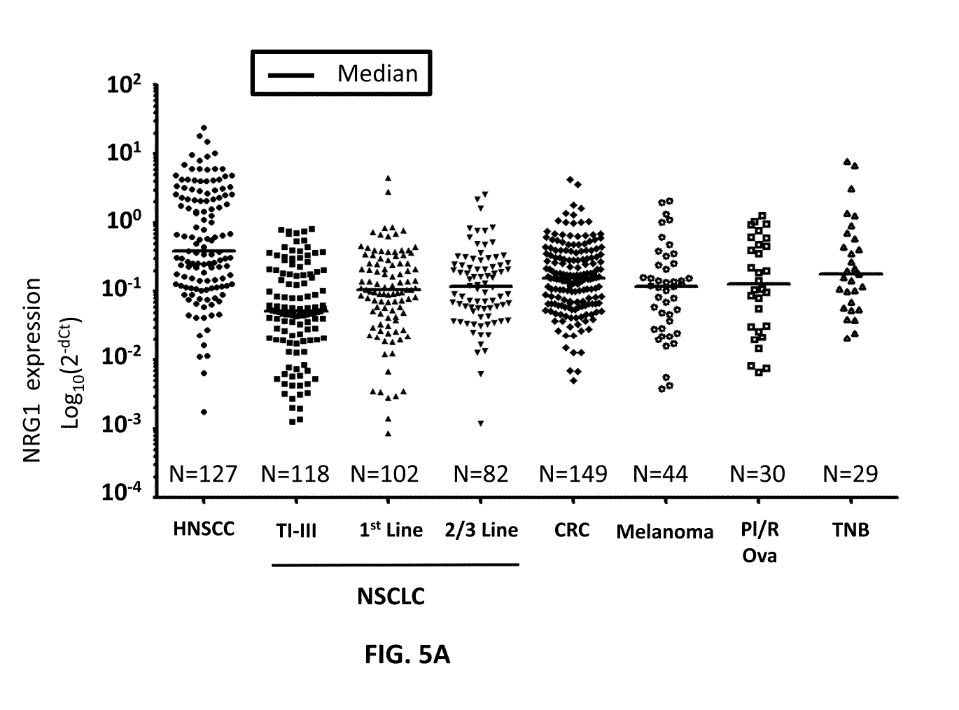

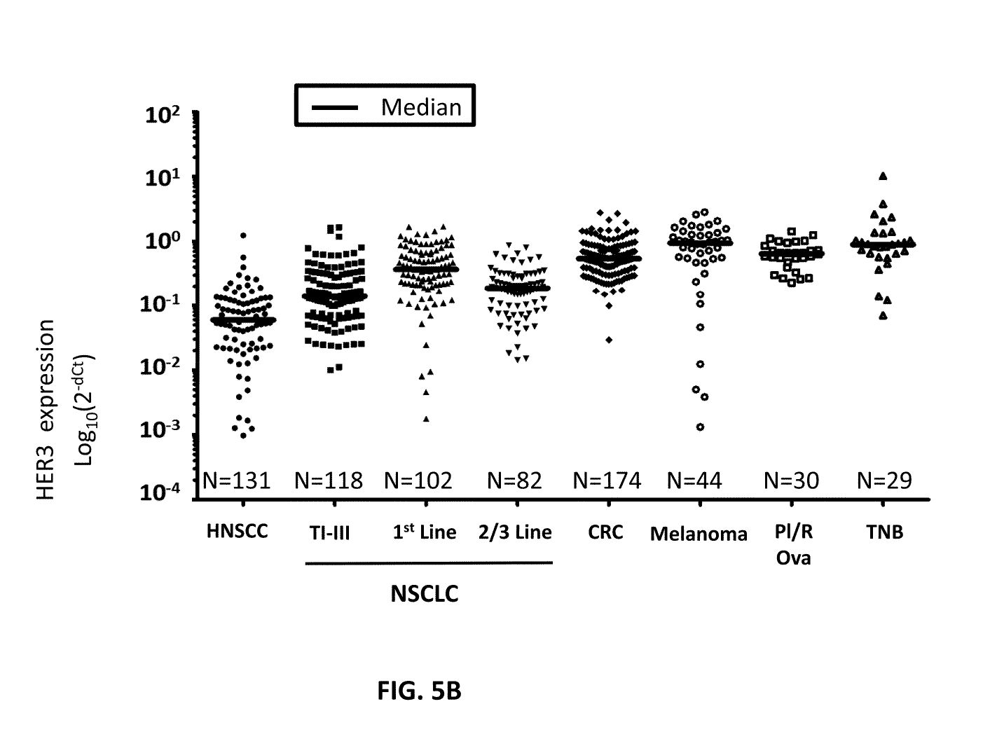

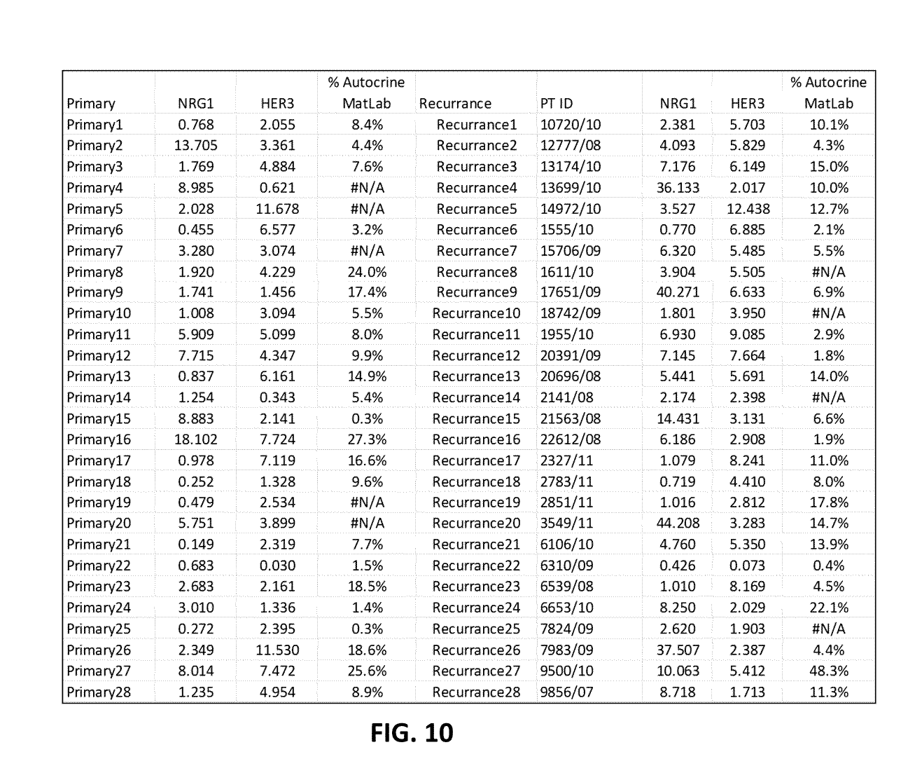

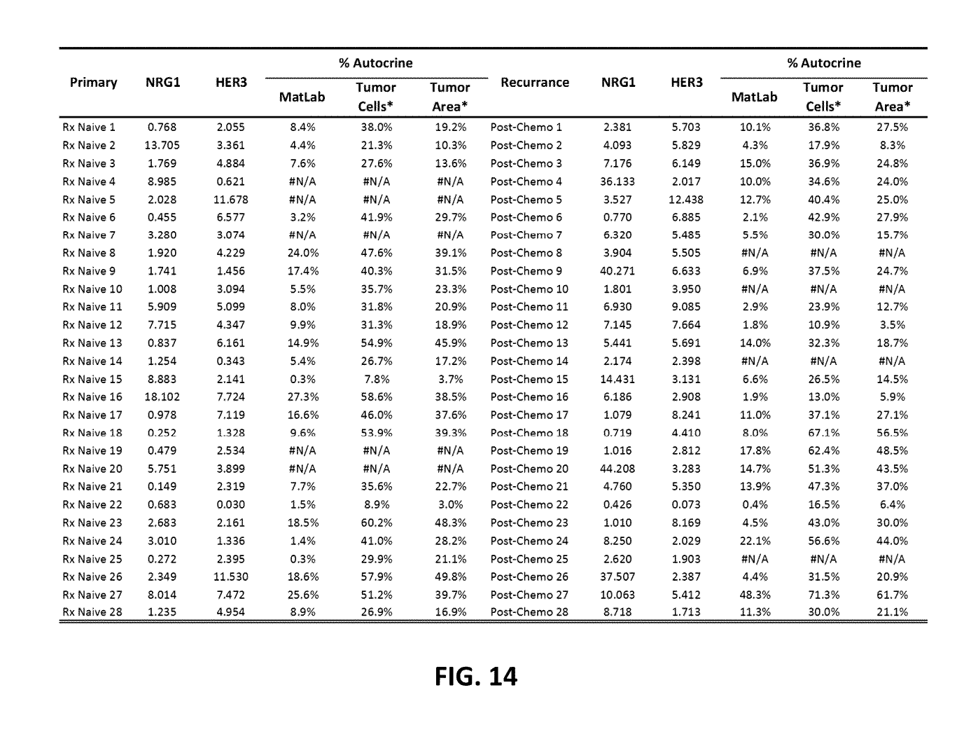

Tissue Specimens: A total of 755 tumor specimens were used in this study: 127 HNSCC (All stages, primary and recurrent), 117 surgically resected NSCLC (Stage I-IV), 102 NSCLCs from patients with untreated metastatic disease (Stage III-IV), 82 NSCLCs from patients who went on to fail front-line standard-of-care but who ultimately received 2 L therapy (Stage III-IV), 29 metastatic platinum refractory ovarian cancers, 149 therapy-naïve metastatic colorectal cancers, 44 primary and metastatic melanomas, and 29 samples from patients with triple-negative breast cancer (stage I-IV). A separate cohort of 20 fresh frozen HNSCCs were used to correlate phosphor-HER3 levels by IP-western with NRG1 transcript. In addition, a separate series of matched samples from a cohort of 28 patients (biopsies from initial diagnosis and at time of first recurrence) with recurrent HNSCC was obtained. (Neither of these two last cohorts was included in FIG. 5) Tables summarizing the available pathologic and demographic variables of these patients and their tumors are shown in FIGS. 10 and 14. All samples were obtained with IRB approval and informed consent.

Tissue Processing for nucleic acids: For RNA extraction tissue sections were submerged in 300 ml RLT buffer (Qiagen) and homogenized using a gentleMACS Octo Dissociator (Miltenyi Biotec). The samples were then split in half for DNA prep (DNAeasy, Qiagen) and RNA prep (TriZol, Invitrogen), as per the manufacturer's instructions.

Fluidigm expression analysis: Gene expression analysis was performed on the cell lines and formlalin-fixed paraffin embedded tumor samples using the BioMark 96×96 gene expression platform (Fluidigm). For the tumors, 2 μl of total RNA was reverse-transcribed to cDNA and pre-amplified in a single reaction using Superscript III/Platinum Taq (Invitrogen) and Pre-amplification reaction mix (Invitrogen). The pre-amplification reaction was performed at a final dilution of 0.05× original Taqman assay concentration (Applied Biosystems). The thermocycling conditions were as follows: 1 cycle of 50° C. for 15 min, 1 cycle of 70° C. for 2 min, then 14 cycles of 95° C. for 15 sec and 60° C. for 4 min.

Pre-amplified cDNA was diluted 1.94-fold and then amplified using Taqman Universal PCR MasterMix (Applied Biosystems) on the BioMark BMK-M-96.96 platform (Fluidigm) according to the manufacturer's instructions. All samples were assayed in triplicate. Two custom-designed reference genes that were previously evaluated for their expression stability across multiple cell lines, fresh-frozen tissue samples, and FFPE tissue samples, AL-1377271 and VPS-33B, were included in the expression panel. A mean of the Ct values for the two reference genes was calculated for each sample, and expression levels of NRG1 and HER3 were determined using the delta Ct (dCt) method as follows: Mean Ct (Target Gene)−Mean Ct (Reference Genes).

Primer/probe sequences for AL137727 (NM_{—}144568 (SEQ ID NO: 10); NM_{—}001100814 (SEQ ID NO: 11) (each sequence represents a splice variant of AL137727)) are as follows:

(SEQ ID NO: 13)
Forward primer: GGCCTCAGTACCCTCAGTCT
(SEQ ID NO: 14)
Reverse primer: AGAGCAGCGGCTGACC
(SEQ ID NO: 15)
FAM Probe: CCCCACAGGACACAAT

Primer/probe sequences for VPS-33B (NM_{—}018668 9(SEQ ID NO: 12)) are as follows:

(SEQ ID NO: 16)
Forward primer: GGCTCGAGACCAGCTCATCTA
(SEQ ID NO: 17)
Reverse primer: GAGATCTGCCTCAATGAATAAATCC
(SEQ ID NO: 18)
FAM Probe: TGGAGCAGCTTCCT

Analysis of distribution of NRG1 expression and cut-off determination: The bimodal distribution of log₁₀ (NRG1) in HNSCC motivates the fitting of the mixture of two normal distributions, one corresponding to the lack of over-expression and the other to over-expression. Let x, denote the log₁₀ NRG1 expression for the ith sample (i=1, . . . , n). The likelihood for the mixture model is:

$$L ( {p}_{k} , {θ}_{k} ) = {\prod }_{i = 1}^{n} {\sum }_{k = 1}^{2} {p}_{k} {f}_{k} ( {x}_{i} ; {μ}_{k ,} {σ}_{k}^{2} ) ,$$

where fₖ is the normal density function of the k^{th} component, and (μₖ, σₖ²) denote the corresponding mean and variance parameters. Maximum likelihood estimates of model parameters were obtained via the EM algorithm (16). Briefly, the E-step computes the conditional probability that the i^{th} sample belongs to the k^{th} component of the mixture given the current parameter estimates. The M-step computes the mixing proportions, means and variances given the current probabilities. The process is then iterated to converge. Posterior probability of component membership was computed for each sample, and the cutoff was selected at the value where the posterior probabilities for the two components were equal. Model fitting was performed using the R package mixtools (17).
Dual Color Chromogenic RNA-ISH: Dual color RNA in situ hybridization was performed by Advanced Cell Diagnostics (Fremont, Calif.). The NRG1 probe set has 31 pairs of oligos (62 total) covering nt 1082-3001 of transcript NM_{—}013964. The ERBB3 probe set is essentially a pool of two probe sets which together cover all of the transcript variants: 20 pairs of oligos (40 total) covering nt 1962-2945 of NM_{—}001982. 14 pairs of oligos (28 total) covering nt 108-899 of NM_{—}001005915. Probes designed against cyclophilin B (PPIB; positive) and the bacterial gene dihydrodipicolinate reductase (DapB; negative) were used as controls. Images were scanned by a Hamamatsu Nanozoomer Digital Slide scanner, running Nanozoomer software, with a 40× objective and 8 bit camera (15, 18). The MatLab (MathWorks, Natick, Mass.) scoring algorithm consists of the following steps: A region of interest with a minimum of 75% tumor cells was manually defined by a pathologist for each section, then a haematoxylin mask was created to identify nuclei, followed by a blue mask (NRG1) and a red mask (HER3) was applied. Individual “cells” were defined by the haematoxylin mask in order to unambiguously separate cells whereupon blue or red dot counts were tabulated for each cell.

Scanned images were also analyzed using Definiens Developer (Munich, AG), using the RGB (red, green and blue) spectra. The same region of interest was used for analysis, as in the MATLAB method. The region was subdivided into tiled regions of approximately 300 um height and width, and analyzed at full resolution. Both color intensity (balance of red, green, and blue intensity values) and object size were used as criteria to distinguish between cell nuclei, NRG1, and HER3, from background. Heavily overlapping nuclei, which were impossible to spectrally separate were excluded from analysis, in order to avoid bias.

Both methods were used to derive each of the following groups of cells: HER3+/NRG+, HER3+/NRG−, HER3−/NRG+, and HER3−/NRG−. Autocrine signal was defined as (HER3+/NRG+)/((HER3+/NRG+)+(HER3+/NRG−)+(HER3−/NRG+)).

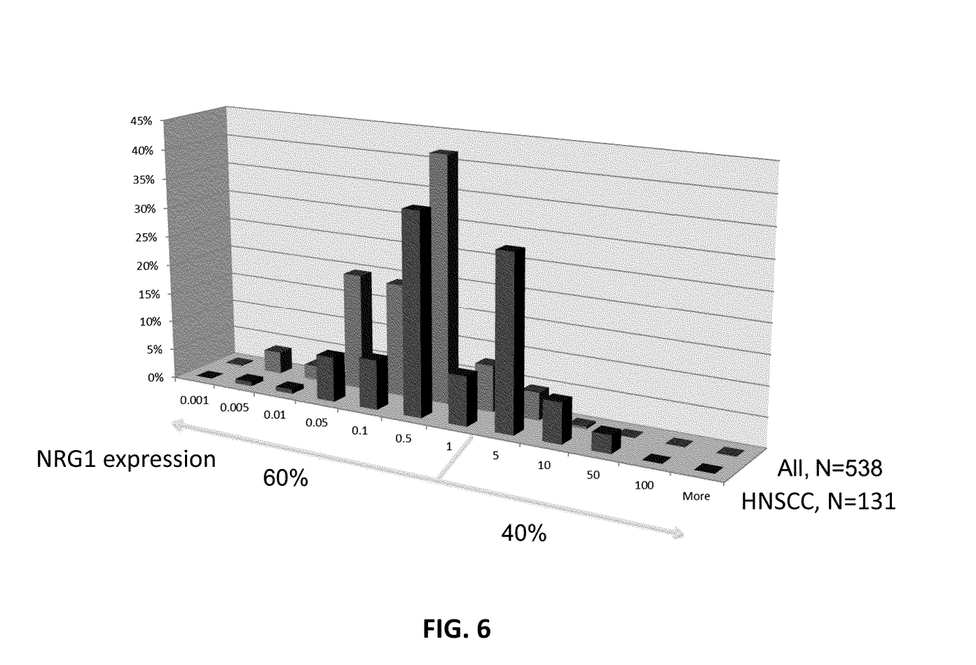

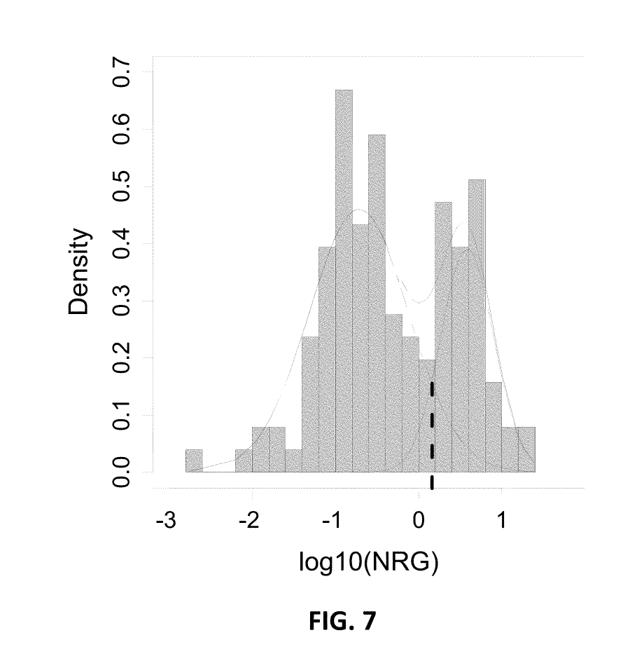

HNSCCs expressed the highest median levels of NRG1 compared to all other tumor types examined (FIG. 5A; FIG. 6). In addition, a significant subset (approx. 40%) of these HNSCCs expressed higher levels of NRG1 than any other tumor type (Mann-Whitney test p<0.0001; FIG. 6). Furthermore, NRG1 expression exhibited a bimodal distribution in HNSCC when plotted on a log₁₀ scale (FIG. 7, FIG. 6). A two-component Gaussian mixture distribution was used to estimate the inflection point between overexpression of NRG1 and lack of overexpression of NRG1, which was found to be at 0.3689 on the logarithm scale, corresponding to approximately 1.50 on the linear scale. The sensitivity and specificity for defining overexpression versus lack of overexpression based on this distribution was 90.8% and 93.4%, respectively.

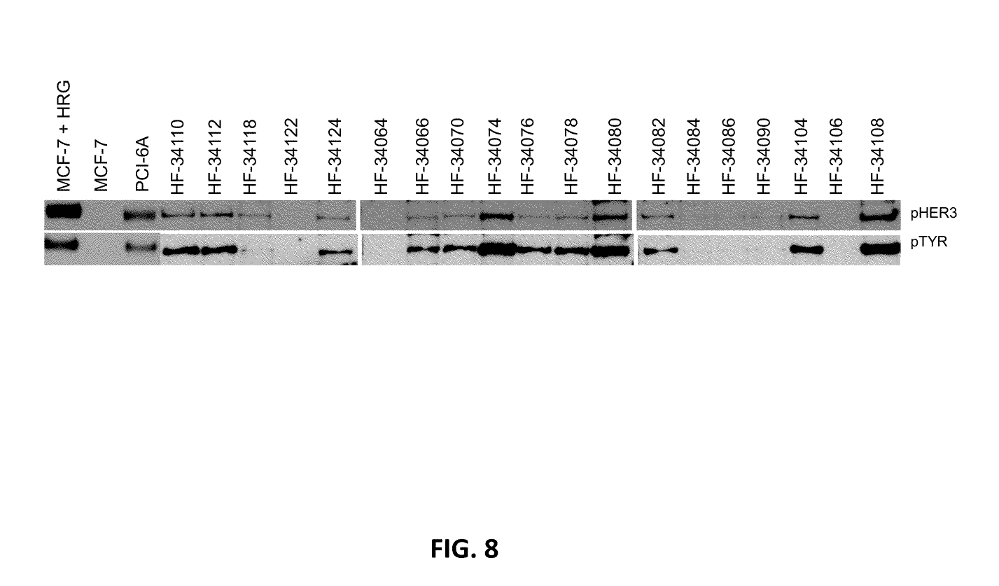

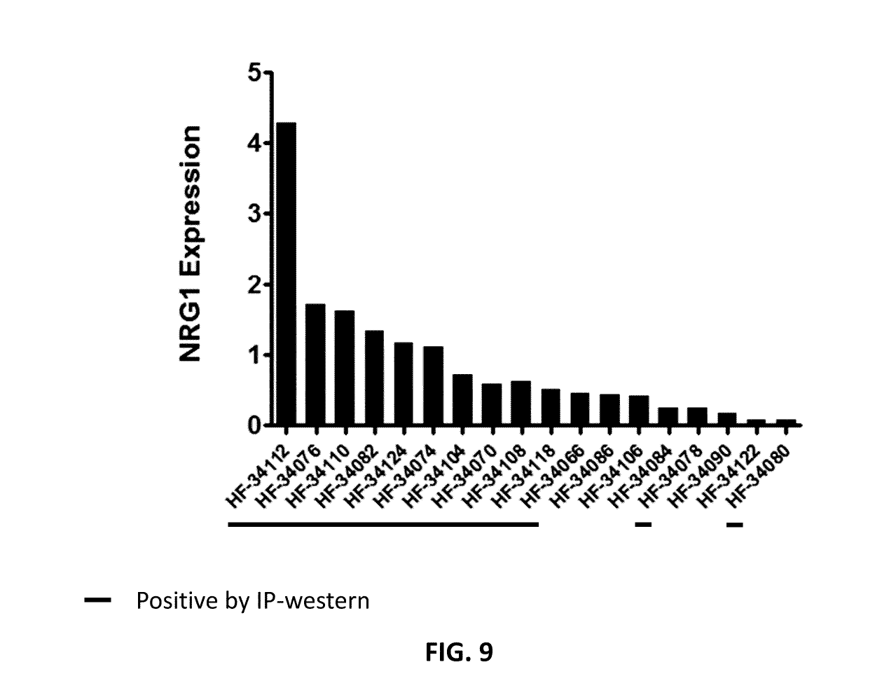

To determine if high NRG1 overexpression was associated with HNSCCs with activated HER3, qRT-PCR was performed for NRG1 and immunoprecipitation of total HER3 followed by western blot for pHER3 and p-Tyrosine in fresh frozen tumor specimens from patients with therapy naïve SCHNN (FIG. 8; FIG. 9; FIG. 10). All tumors with high NRG1 expression (at or near the low-point within the bimodal distribution) were positive for pHER3 by IP-western; conversely 5/7 tumors with the lowest expression of NRG1 were negative for pHER3 (two-tailed sign test, P=0.0386).

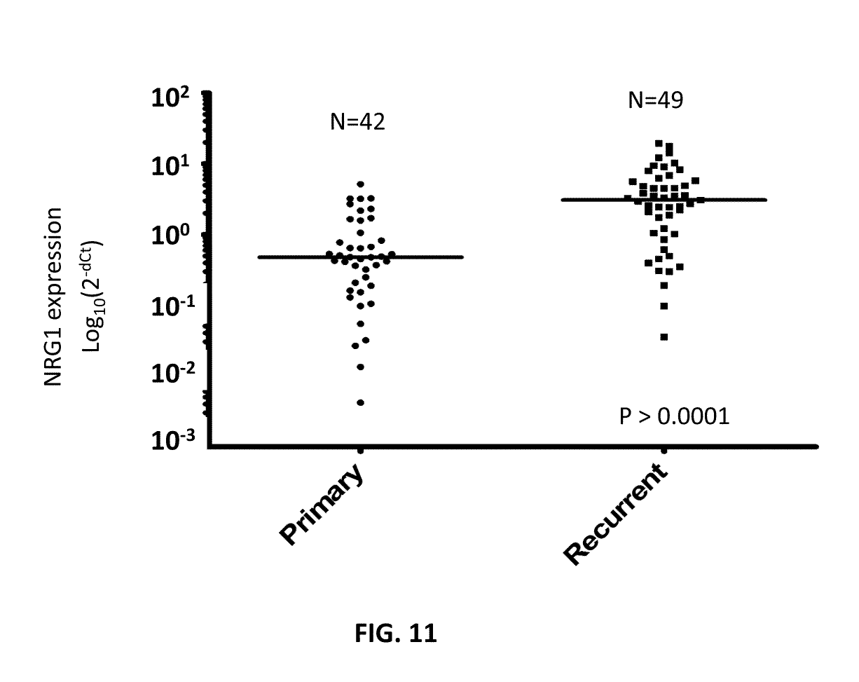

To further characterize HNSCC patient populations with tumors that show high NRG1 expression, tumor samples were obtained from patients with surgically resectable HNSCC as well as tumor samples from patients with recurrent disease. NRG1 expression was higher in the recurrent setting compared to primary resectable disease (FIG. 11). These findings could not be explained by differences in site of origin, HPV status, or any other available clinicopathological information.

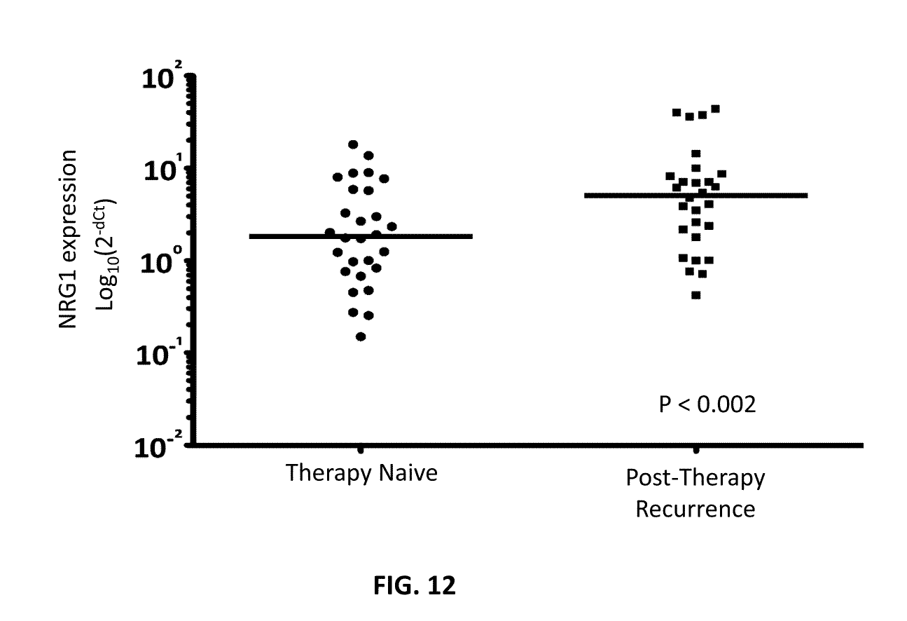

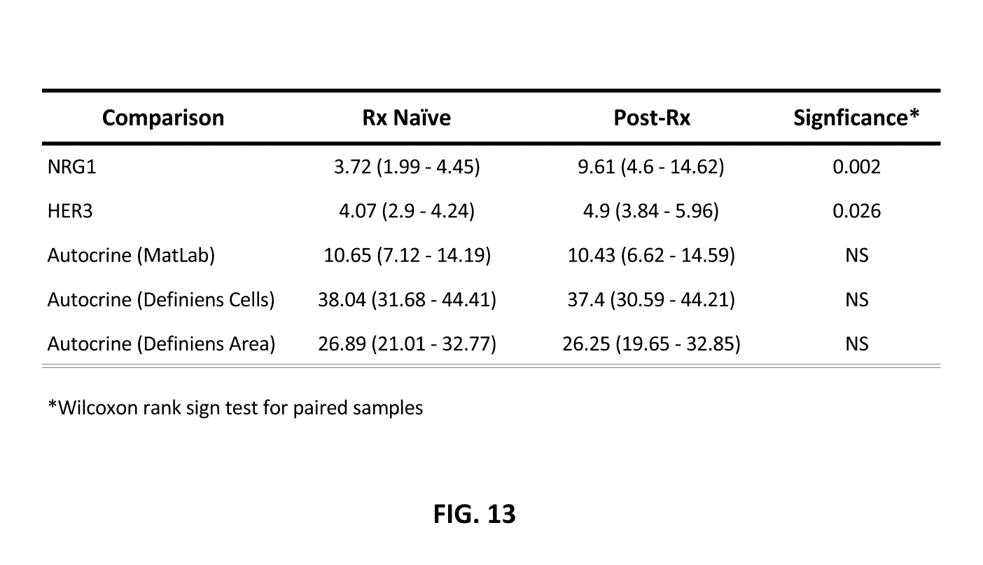

The finding that NRG1 expression may differ in primary versus recurrent disease could suggest that NRG1 expression increases as a consequence of prior therapy, or because high NRG1 expression is a prognostic factor associated with an increased likelihood of recurrence in patients with HNSCC. To explore this question further, a series of patient matched primary tumors and relapse specimens were obtained and compared NRG1 expression in both cohorts by qRT-PCR. As shown in FIG. 12; FIG. 13; FIG. 14), NRG1 expression is significantly higher in the recurrent setting compared to the primary disease in matched patient specimens (Wilcoxon signed rank test; P=0.002).

Preclinical data from a recent report suggested that identifying tumors with autocrine biology may be important in predicting response to agents targeting HER family receptors (11). To determine the extent of autocrine versus paracrine expression of HER3 and NRG1 expression in HNSCCs, a dual-color chromogenic RNA in situ hybridization assay designed to evaluate HER3 and NRG1 transcript location and abundance in clinically relevant FFPE was used.

Qualitative examination of benign squamous epithelium suggested that expression of NRG1 was limited to the basal layer, whereas HER3 expression was only seen focally in normal stratified squamous epithelium. In contrast, in the spiny layer, only HER3 expression was observed. A similar pattern of expression was seen in pseudostratified upper respiratory tract epithelium where NRG1 expression was limited to the basal layer and HER3 expression was restricted to the upper layers of the epithelium. At the single cell level in benign tissue, absent a source of NRG1, most cells and tissues express relatively consistent levels of HER3, whereas in the presence of NRG1, there is a gradient of HER3 expression that increases the farther away the cells are from the source of NRG1. More generally, there is an inverse relationship between the expression of HER3 and NRG1 both at the level of individual cells as well as in the spatial orientation of cells that express either of these two transcripts

Clear evidence of paracrine expression of NRG1 and HER3 in some well-differentiated head and neck squamous cell carcinomas was found, recapitulating the expression patterns seen in normal tissue. As used herein, autocrine is defined as the co-expression of NRG1 and HER3 in the same cell, whereas paracrine expression is defined as mutually exclusive expression of either NRG1 or HER3 in adjacent cells.

In other more poorly differentiated squamous cell carcinomas the relationship between basal cell specific expression of NRG1 and spiny cell specific expression of HER3 was lost, consistent with the more disordered architecture, with evidence for both autocrine and paracrine expression pattern. Finally, cases where the majority of HER3/NRG1 expressing cells appeared to be autocrine were identified.

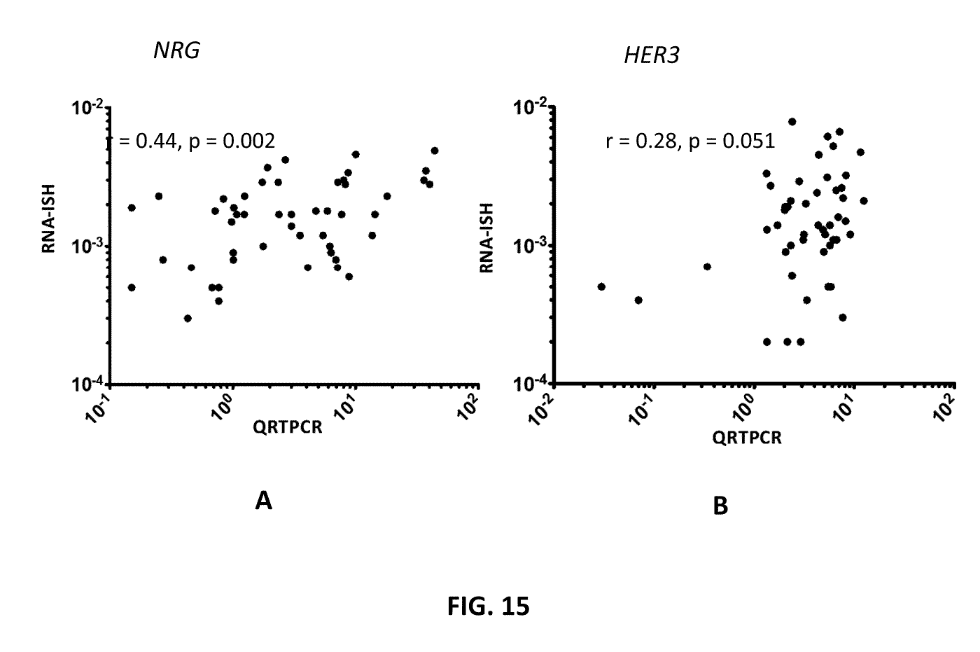

To determine if high NRG1 expression was specifically associated with either autocrine or paracrine expression and to ensure that the increase in NRG1 expression observed between primary treatment naïve specimens and their counterpart post-treatment sections was tumor derived, qRTPCR was compared with RNA-ISH for NRG1 and HER3 in the same samples represented in FIG. 12. Overall, there was a strong, significant positive Spearman correlation between qRTPCR and RNA-ISH for NRG1 (r=0.4; p=0.002) and a lower, near significant, positive correlation between qRTPCR and RNA-ISH for HER3 (p=0.2; p=0.051) (FIGS. 15A & B).

Three different quantitative imaging algorithms were used to identify autocrine cells (as described in this Example above). While each method differed in the relative proportion of autocrine component for each tumor, the methods ranked the different tumors similarly (Spearman r: ˜0.75-0.96 for all pairwise combinations, p<0.0001 in all cases) between MatLab and each of the Definiens-based approaches. This suggests that the algorithms were differentiating autocrine and paracrine phenotypes in a similar way, albeit with potentially different sensitivities. Unlike NRG1 expression, there was no difference in the relative contribution of autocrine expression between therapy naïve and post-chemotherapy specimens (FIG. 13; FIG. 14). Furthermore, there was very little association between levels of NRG1 expression by qRTPCR or RNA-ISH and the proportion of cells with autocrine expression of NRG1 and HER3 within the tumors before or after chemotherapy (Spearman r: 0.26 (p=0.22), 0.03 (p=0.91), respectively).

This data shows that a substantial subset of HNSCCs expressed significantly higher levels of NRG1 compared to all other tumor types examined NRG1 expression has a unique bimodal distribution in HNSCC, with approximately 40% of HNSCC tumors expressing higher levels of NRG1 than all other tumor types. This pattern of expression is reminiscent of HER2 expression in breast cancer, where HER2 expression levels are at least an order of magnitude higher in HER2 positive breast cancers compared to other types of breast cancers. However, unlike HER2 in breast and gastric cancers, NRG1 overexpression does not appear to be a function of gene amplification (20, 21).

Additionally, high NRG1 expression was associated with pHER3 possibly indicating active HER3 signaling in these particular HNSCC tumors. pHER3 was detectable in all cases where NRG1 was expressed at or near the low point of the bimodal distribution in the HNSCC patients. In some cases pHER3 was detected in samples with NRG1 levels that were somewhat below the low point of the distribution. The low point of the bimodal distribution was similar in multiple independent data sets, potentially reflecting biologically distinct populations of HNSCC patients and providing a cut-off for identifying patients with HNSCC that might benefit from HER3-directed therapeutic intervention.

Importantly, two of the lowest NRG1 expressing tumors also had detectable pHER3. It has been shown that NRG-independent phosphorylation of HER3 can occur through heterodimerization with EGFR or other RTKs such as c-Met (1). Patients with tumors exhibiting NRG-independent activation of HER3 are unlikely to benefit from HER3 directed therapies and thus it is important to note that using NRG1 as a predictive marker would effectively exclude this patient population (12).

Furthermore, NRG1 expression is higher in recurrent tumor specimens compared to matched and unmatched primary tumors. These findings, taken together with the data above, suggest that NRG1 expression may be both predictive of response to HER3 inhibitors and prognostic for recurrence of HNSCC.

Accordingly. NRG1 expression levels define a statistically and biologically distinguishable subset of HNSCC patients. High-level expression of NRG1 is associated with constitutive activation of HER3 in HNSCC and thus defines an actionable biomarker for drugs that inhibit this important oncogene.

### Example 5

### Neuregulin 1 Expression Predicts Response to Treatment in Patients Enrolled in a Phase I Study of MEHD7945A

### Summary of Clinical Data

MEHD7945A has been initially studied in an open-label, multicenter, Phase I study (DAF4873g) to evaluate its safety and tolerability, pharmacokinetics, pharmacodynamics, and/or anti-tumor activity when administered by IV infusion every 2 weeks (q2w) in patients with refractory or relapsed epithelial tumors. The study consists of 3+3 dose-escalation cohorts with a 28-day window to evaluate dose-limiting toxicity (DLT), as well as enrollment of multiple expansion cohorts at the recommended Phase II dose.

Cancer types the patients enrolled in this study exhibited included HNSCC, colorectal cancer, lung cancer, breast cancer, pancreatic cancer, ovarian cancer and liver/biliary cancer.

Dose levels ranging from 1 to 30 mg/kg were evaluated. Drug clearance decreased in a dose-dependent fashion, approaching linearity at doses >10 mg/kg and suggesting that MEHD7945A is subject to target-mediated clearance similar to that seen with monospecific anti-EGFR antibodies. A dose of 1100 mg on a q2w schedule is expected to provide the weekly efficacious exposure determined in xenograft models in >95% of patients.

Two patients with HNSCC treated with MEHD7945A at 14 mg/kg q2w have ongoing partial responses, with durations of 10.7 and 3.9 weeks. Thirteen of 66 patients had experienced stable disease as their best response, with stable disease maintained for ≧4 months in 3 patients, including 1 patient with KRAS wild-type mCRC, previously refractory to FOLFIRI+cetuximab.

Tumor-associated pharmacodynamic effects were observed at doses of 10 to 30 mg/kg in a total 15 patients, 11 of whom had previously received EGFR-targeting therapy. Decreased phosphorylation of tumor S6, PRAS40, and ERK was observed in serial biopsies from 6 of 17 patients with evaluable tissue samples, and metabolic responses (≧20% decrease in fluorodeoxyglucose [FDG] uptake by positron emission tomography [PET] scan) were observed in 10 of 56 patients with PET-avid disease at baseline. Tumor types in which these changes were observed include CRC, NSCLC, HNSCC, and ovarian, breast, and anal cancer.

Samples from the patients were analyzed for NRG1 expression.

As noted above, two patients with HNSCC in this Phase I study exhibited partial responses to treatment with the MEHD7945A bispecific antibody. Patient 1 was diagnosed in 2007 with HNSCC of the larynx, prior therapies include chemoradiation, 3× cetuximab±chemotherapy with best response of SD.

Patient 1 had a confirmed partial response after treatment with MEHD7945A (14 mg/kg IV). The partial response was indicated by reduction in tumor size based on CT analysis and using the applicable criteria provided by the Response Evaluation Criteria in Solid Tumors (RECIST) guidelines. Patient 1 also exhibited clinical improvement (less pain, improved phonation). Patient 2 was diagnosed in 1994 with HNSCC of the tongue, recently metastatic to the lung. Prior therapies include multiple surgeries and chemoradiation. Patient 2 had a confirmed partial response indicated by reduction in tumor size based on CT analysis after treatment with MEHD7945A (14 mg/kg IV q2w) and had clincial improvement (regained ability to swallow).

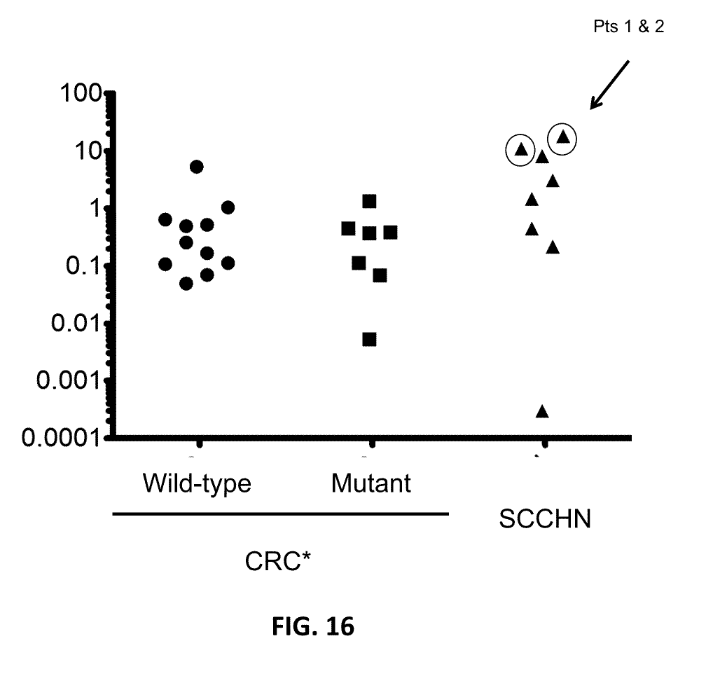

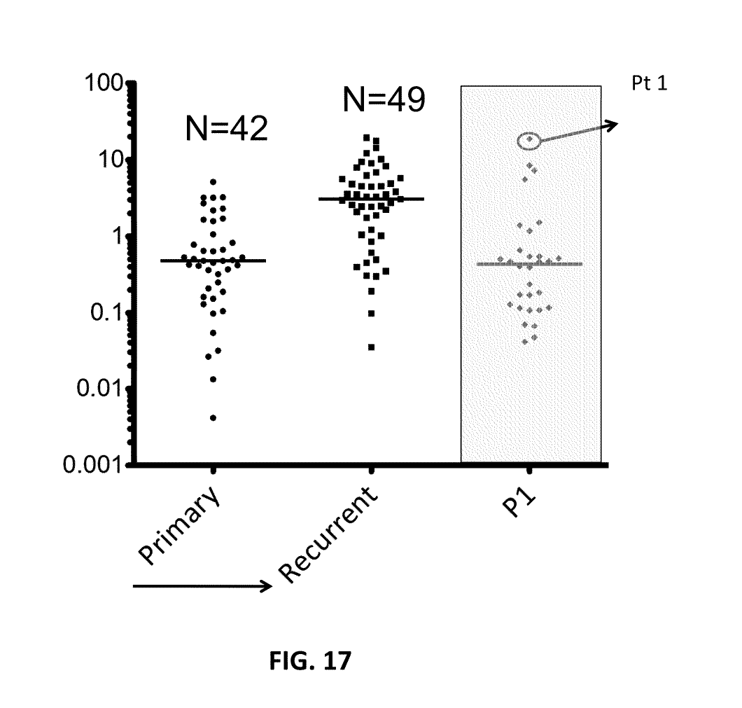

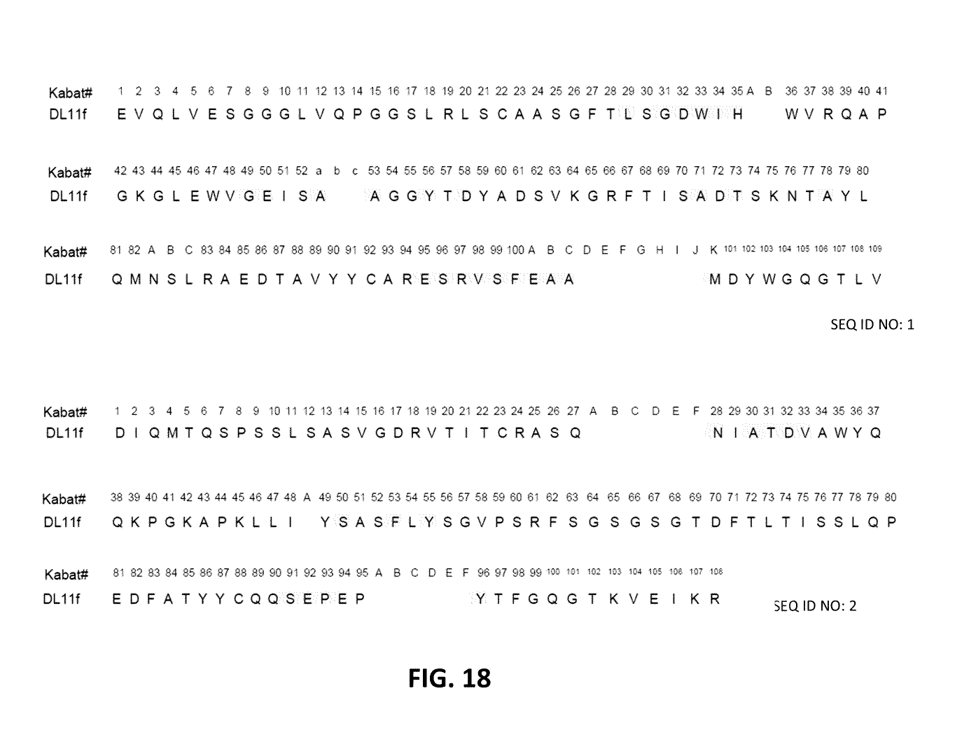

These two patients had cancers which exhibited the highest level of NRG1 among the study group. (FIG. 16). Furthermore, NRG1 expression levels in recurrent HNSCC were determined to be higher than NRG1 expression levels in primary HNSCC. FIG. 17 shows the result of this assay and compares the NRG1 expression level of Patient 1 from the Phase 1 trial.

### REFERENCES

1. Engelman J A, Zejnullahu K, Mitsudomi T, Song Y, Hyland C, Park J O, et al. MET amplification leads to gefitinib resistance in lung cancer by activating ERBB3 signaling. Science. 2007; 316:1039-43.
2. Howlader N, Noone A M, Krapcho M, Neyman N, Aminou R, Altekruse S F, et al. SEER Cancer Statistics Review, 1975-2009 (Vintage 2009 Populations). SEER Cancer Statistics Review, 1975-2009 (Vintage 2009 Populations). 2012 [cited; Available from:
3. Lefebvre J L. Current clinical outcomes demand new treatment options for HNSCC. Ann Oncol. 2005; 16 Suppl 6:vi7-vi12.
4. Leemans C R, Braakhuis B J, Brakenhoff R H. The molecular biology of head and neck cancer. Nat Rev Cancer. 2011; 11:9-22.
5. Morgan S, Grandis J R. ErbB receptors in the biology and pathology of the aerodigestive tract. Exp Cell Res. 2009; 315:572-82.
6. Kalyankrishna S, Grandis J R. Epidermal growth factor receptor biology in head and neck cancer. J Clin Oncol. 2006; 24:2666-72.
7. Bonner J A, Harari P M, Giralt J, Azarnia N, Shin D M, Cohen R B, et al. Radiotherapy plus Cetuximab for Squamous-Cell Carcinoma of the Head and Neck. New England Journal of Medicine. 2006; 354:567-78.
8. Cohen E E W, Davis D W, Karrison T G, Seiwert T Y, Wong S J, Nattam S, et al. Erlotinib and bevacizumab in patients with recurrent or metastatic squamous-cell carcinoma of the head and neck: a phase I/II study. The Lancet Oncology. 2009; 10:247-57.
9. Stewart J S, Cohen E E, Licitra L, Van Herpen C M, Khorprasert C, Soulieres D, et al. Phase III study of gefitinib compared with intravenous methotrexate for recurrent squamous cell carcinoma of the head and neck [corrected]. J Clin Oncol. 2009; 27:1864-71.
10. del Campo J M, Hitt R, Sebastian P, Carracedo C, Lokanatha D, Bourhis J, et al. Effects of lapatinib monotherapy: results of a randomised phase II study in therapy-naive patients with locally advanced squamous cell carcinoma of the head and neck. Br J. Cancer. 2011; 105:618-27.
11. Wilson T R, Lee D Y, Berry L, Shames D S, Settleman J. Neuregulin-1-mediated autocrine signaling underlies sensitivity to HER2 kinase inhibitors in a subset of human cancers. Cancer Cell. 2011; 20:158-72.
12. Schaefer G, Haber L, Crocker L M, Shia S, Shao L, Dowbenko D, et al. A two-in-one antibody against HER3 and EGFR has superior inhibitory activity compared with monospecific antibodies. Cancer Cell. 2011; 20:472-86.
13. Holmes W E, Sliwkowski M X, Akita R W, Henzel W J, Lee J, Park J W, et al. Identification of heregulin, a specific activator of p185erbB2. Science. 1992; 256:1205-10.
14. Sliwkowski M X, Schaefer G, Akita R W, Lofgren J A, Fitzpatrick V D, Nuijens A, et al. Coexpression of erbB2 and erbB3 proteins reconstitutes a high affinity receptor for heregulin. J Biol. Chem. 1994; 269:14661-5.
15. Cervantes-Ruiperez A, Juric D, Hidalgo M, Messersmith W A, Blumenschein G R, Baselga J, et al. A phase I study of MEHD7945A (MEHD), a first-in-class HER3/EGFR dual-action antibody, in patients (pts) with refractory/recurrent epithelial tumors: Expansion cohorts. ASCO Meeting Abstracts. 2012; 30:2568.
16. Dempster A P, Laird N M, Rubin D B. Maximum Likelihood from Incomplete Data Via Em Algorithm. J Roy Stat Soc B Met. 1977; 39:1-38.
17. Benaglia T, Chaiveau D, Hunter D R, Young D. mixtools: An R Package for Analyzing Finite Mixture Models. Journal of Statistical Software. 2009; 32:1-29.
18. Shames D S, Stern H, Walter K, Jiang B, Fu L, Do A, et al. Identification of head and neck cancers (SCCHN) that may respond to dual inhibition of EGFR and HER3 signaling. ASCO Meeting Abstracts. 2012; 30:5575.
19. Walter K, Holcomb T, Januario T, Du P, Evangelista M, Kartha N, et al. DNA methylation profiling defines clinically relevant biological subsets of non-small cell lung cancer. Clin Cancer Res. 2012; 18:2360-73.
20. Agrawal N, Frederick M J, Pickering C R, Bettegowda C, Chang K, Li R J, et al. Exome sequencing of head and neck squamous cell carcinoma reveals inactivating mutations in NOTCH1. Science. 2011; 333:1154-7.
21. Stransky N, Egloff A M, Tward A D, Kostic A D, Cibulskis K, Sivachenko A, et al. The mutational landscape of head and neck squamous cell carcinoma. Science. 2011; 333:1157-60.

What is claimed is:

1. A method of treating a type of cancer in a patient comprising administering a therapeutically effective amount of a HER3 inhibitor to the patient, wherein the patient, prior to administration of the HER3 inhibitor, was diagnosed with a cancer which overexpresses NRG1.

2. The method of claim 1, wherein the patient was diagnosed with a cancer expressing NRG1 at a level higher than the median level for NRG1 expression in the cancer type.

3. The method of claim 2, wherein the patient was diagnosed with a cancer expressing NRG1 at a level which is the 60th percentile or higher for NRG1 expression in the cancer type.

4. The method of claim 2, wherein the patient was diagnosed with a cancer expressing NRG1 at a level which is the 75th percentile or higher for NRG1 expression in the cancer type.

5. The method of claim 2, wherein the patient was diagnosed with a cancer expressing NRG1 at a level which is the 80th percentile or higher for NRG1 expression in the cancer type.

6. The method of claim 1, wherein the type of cancer is one which exhibits a bimodal expression profile consisting of an overexpression mode and a lack of overexpression mode.

7. The method of claim 1, wherein the type of cancer exhibits autocrine neuregulin-induced signaling.

8. The method of claim 1, wherein the type of cancer is head and neck squamous cell carcinoma (HNSCC).

9. The method of claim 1, wherein the HER3 inhibitor inhibits NRG1 binding to HER3.

10. The method of claim 1, wherein the HER3 inhibitor is an antibody.

11. The method of claim 1, wherein the HER3 inhibitor is a bispecific HER3/EGFR inhibitor.

12. The method of claim 11, wherein the bispecific HER3/EGFR inhibitor is a bispecific antibody which comprises an antigen binding domain that specifically binds to HER3 and EGFR.

13. The method of claim 12, wherein the antigen binding domain that specifically binds to HER3 and EGFR comprises
a HVR-H1 comprising the amino acid sequence LSGDWIH (SEQ ID NO: 3),
a HVR-H2 comprising the amino acid sequence VGEISAAGGYTD (SEQ ID NO: 4),
a HVR-H3 comprising the amino acid sequence ARESRVSFEAAMDY (SEQ ID NO: 5), and
a HVR-L1 comprising the amino acid sequence NIATDVA (SEQ ID NO: 6),
a HVR-L2 comprising the amino acid sequence SASF (SEQ ID NO: 7), and
a HVR-L3 comprising the amino acid sequence SEPEPYT (SEQ ID NO: 8).

14. The method of claim 13, wherein the antigen binding domain that specifically binds to HER3 and EGFR comprises a heavy chain variable domain having at least 95% sequence identity to the amino acid sequence of SEQ ID NO: 1 and a light chain variable domain having at least 95% sequence identity to the amino acid sequence of SEQ ID NO: 2.

15. The method of claim 12, wherein the antigen binding domain that specifically binds to HER3 and EGFR comprises a heavy chain variable domain of SEQ ID NO: 1 and a light chain variable domain of SEQ ID NO: 2.

16. The method of claim 1, wherein the diagnosis comprised determining the expression level of NRG1 in a sample from the patient's cancer and quantifying the expression level of NRG1 in the sample relative to the expression level of one or both of AL-137727 and VPS33B in the sample.

17. A method of treating head and neck squamous cell carcinoma (HNSCC) in a patient comprising administering a therapeutically effective amount of a bispecific HER3/EGFR inhibitor to the patient, wherein the patient, prior to administration of the bispecific HER3/EGFR inhibitor, was diagnosed with a HNSCC which overexpresses NRG1.

18. The method of claim 17, wherein the bispecific HER3/EGFR inhibitor is a bispecific antibody which comprises an antigen binding domain that specifically binds to HER3 and EGFR.

19. The method of claim 17, wherein the antigen binding domain that specifically binds to HER3 and EGFR comprises
a HVR-H1 comprising the amino acid sequence LSGDWIH (SEQ ID NO: 3),
a HVR-H2 comprising the amino acid sequence VGEISAAGGYTD (SEQ ID NO: 4), and
a HVR-H3 comprising the amino acid sequence ARESRVSFEAAMDY (SEQ ID NO: 5), and
a HVR-L1 comprising the amino acid sequence NIATDVA (SEQ ID NO: 6),
a HVR-L2 comprising the amino acid sequence SASF (SEQ ID NO: 7), and
a HVR-L3 comprising the amino acid sequence SEPEPYT (SEQ ID NO: 8).

20. The method of claim 19, wherein the antigen binding domain that specifically binds to HER3 and EGFR comprises a heavy chain variable domain having at least 95% sequence identity to the amino acid sequence of SEQ ID NO: 1 and a light chain variable domain having at least 95% sequence identity to the amino acid sequence of SEQ ID NO: 2.

21. The method of claim 18, wherein the antigen binding domain that specifically binds to HER3 and EGFR comprises a heavy chain variable domain of SEQ ID NO: 1 and a light chain variable domain sequence of SEQ ID NO: 2.

22. The method of claim 17, wherein the diagnosis comprised determining the expression level of NRG1 in a sample from the patient's HNSCC and quantifying the expression level of NRG1 in the sample relative to the expression level of one or both of AL-137727 and VPS33B in the sample.

23. A method for selecting a therapy for a patient with a type of cancer which exhibits autocrine neuregulin-induced signaling comprising determining neuregulin 1 (NRG1) expression in a cancer sample from the patient and selecting a HER3 inhibitor for therapy if the cancer sample overexpresses NRG1.

24. The method of claim 23, wherein the cancer sample expresses NRG1 at a level higher than the median level for NRG1 expression in the cancer type.

25. The method of claim 24, wherein the cancer sample expresses NRG1 at a level which is the 60th percentile or higher for NRG1 expression in the cancer type.

26. The method of claim 24, wherein the cancer sample expresses NRG1 at a level which is the 75th percentile or higher for NRG1 expression in the cancer type.

27. The method of claim 24, wherein the cancer sample expresses NRG1 at a level which is the 80th percentile or higher for NRG1 expression in the cancer type.

28. The method of claim 23, wherein the type of cancer is one which exhibits a bimodal expression profile consisting of an overexpression mode and a lack of overexpression mode.

29. The method of claim 23, wherein the type of cancer is head and neck squamous cell carcinoma (HNSCC)

30. The method of claim 23, wherein the HER3 inhibitor inhibits NRG1 binding to HER3.

31. The method of claim 23, wherein the HER3 inhibitor is an antibody.

32. The method of claim 31, wherein the HER3 inhibitor is a bispecific HER3/EGFR inhibitor.

33. The method of claim 32, wherein the bispecific HER3/EGFR inhibitor is a bispecific antibody which comprises an antigen binding domain that specifically binds to HER3 and EGFR.

34. The method of claim 33, wherein the antigen binding domain that specifically binds to HER3 and EGFR comprises
a HVR-H1 comprising the amino acid sequence LSGDWIH (SEQ ID NO: 3),
a HVR-H2 comprising the amino acid sequence VGEISAAGGYTD (SEQ ID NO: 4), and
a HVR-H3 comprising the amino acid sequence ARESRVSFEAAMDY (SEQ ID NO: 5), and
a HVR-L1 comprising the amino acid sequence NIATDVA (SEQ ID NO: 6),
a HVR-L2 comprising the amino acid sequence SASF (SEQ ID NO: 7), and
a HVR-L3 comprising the amino acid sequence SEPEPYT (SEQ ID NO: 8).

35. The method of claim 34, wherein the antigen binding domain that specifically binds to HER3 and EGFR comprises a heavy chain variable domain having at least 95% sequence identity to the amino acid sequence of SEQ ID NO: 1 and a light chain variable domain having at least 95% sequence identity to the amino acid sequence of SEQ ID NO: 2.

36. The method of claim 33, wherein the bispecific antibody comprises a heavy chain variable domain of SEQ ID NO: 1 and a light chain variable domain sequence of SEQ ID NO: 2.

37. The method of claim 23, wherein NRG1 expression has been determined using polymerase chain reaction (PCR).

38. The method of claim 37, wherein the PCR is quantitative real time polymerase chain reaction (qRT-PCR).

39. The method of claim 23, wherein NRG1 expression has been determined using IHC.

40. The method of claim 23, wherein the determining the NRG1 expression in the HNSCC sample comprises determining the expression level of NRG1 in the sample and quantifying the expression level of NRG1 in the sample relative to the expression level of one or both of AL-137727 and VPS33B in the sample.

41. The method of claim 23, further comprising administering a therapeutically effective amount of the HER3 inhibitor to the patient.

42. A method of quantifying NRG1 expression level in a cancer sample comprising:
determining the expression level of NRG1 in the sample, and
quantifying the expression level of NRG1 in the sample relative to the expression level of one or both of AL-137727 and VPS33B in the sample.

43. The method of claim 42, wherein the cancer exhibits autocrine neuregulin-induced signaling.

44. The method of claim 42, wherein the cancer is head and neck squamous cell carcinoma (HNSCC).

45. The method of claim 42, wherein the expression level of NRG1 and the expression level of the one or both of AL-137727 and VPS33B is determined using polymerase chain reaction (PCR).

46. The method of claim 45, wherein the PCR is quantitative real time polymerase chain reaction (qRT-PCR).

47. The method of claim 42, wherein the expression level of NRG1 and the expression level of the one or both of AL-137727 and VPS33B is determined using immunohistochemistry (IHC).
